(19) United States
(12) Patent Application Publication
(10) Pub. No.: US 2006/0264497 A1
(43) Pub. Date: Nov. 23, 2006

(54) Diindolylmethane-based compositions and methods of use thereof for promoting oral mucosal and bone health

(76) Inventor: Michael A. Zeligs, Boulder, CO (US)

Correspondence Address: JONES DAY, 222 EAST 41ST ST, NEW YORK, NY, 10017, US

(21) Appl. No.: 11/392,840

(22) Filed: Mar. 28, 2006

Related U.S. Application Data
(60) Provisional application No. 60/666,255, filed on Mar. 28, 2005.
(60) Provisional application No. 60/776,122, filed on Feb. 22, 2006.

(51) Int. Cl. A61K 31/404

(52) U.S. Cl. 514/414

(57) ABSTRACT

The present invention includes compositions and methods for the treatment and prevention of oral mucosal disorders and for promotion of bone health. In particular, the present invention describes new therapeutic and preventative uses for 3,3′-diindolylmethane (DIM), or a DIM-related indole, alone or in combination with anti-inflammatory agents and/or antibacterial agents, to treat oral mucosal disorders and promote bone health. The compositions of the invention are used to prevent and reverse oral mucosal disorders and bone loss (osteopenia and osteoporosis) associated with aging and chronic inflammation. Oral mucosal disorders include Periodontitis, gingivitis and related oral mucosal inflammation. Formulations of the compositions of the invention include capsules, tablets, toothpastes, oral gels, mouthwashes, mouth rinses, lozenges, chewing gum, dental floss, and dental topical formulations, and fortified foods.

This application claims the benefit of U.S. Provisional Application No. 60/666,255, filed Mar. 28, 2005, and U.S. Provisional Application No. 60/776,122, filed Feb. 22, 2006, each of which is incorporated by reference in its entirety.

### 1 FIELD OF THE INVENTION

The invention relates to Diindolylmethane (DIM) based methods and compositions to promote oral mucosal and bone health. In particular, the invention relates to the treatment and prevention of mucosal disorders of the oral cavity, including periodontitis and related forms of gingival disease using diindolylmethane (DIM) or a DIM-related indole. DIM and DIM-related indoles are used alone or in combination with other anti-inflammatory compounds and/or anti-bacterial compounds to prevent and treat a spectrum of oral mucosal pathology including calculus accumulation (dental plaque), apthous ulcers, oral malodor (halitosis), gingivitis, periodontitis, alveolar bone loss, and loss of teeth. The invention also relates to methods and compositions for the promotion of general bone health using diindolylmethane (DIM) or a DIM-related indole. DIM, and DIM-related indoles, are used alone or in combination with selected phytoestrogen compounds, especially genistein and related isoflavones, to promote bone health and to prevent and treat aging-related bone loss (osteopenia and osteoporosis). The methods and compositions of the present invention are further used to treat and prevent bone loss associated with chronic inflammatory conditions, particularly Rheumatoid Arthritis and Systemic Lupus Erythematosus.

### 2 BACKGROUND OF THE INVENTION

### 2.1 Inflammation in Oral Mucosal Disorders and Bone Loss

Oral mucosa and bone tissue are subject to common immune system responses to chronic inflammation. Both oral mucosa and bone tissue attract inflammation-associated white blood cells. In the oral cavity, microbial growth generates inflammation which attracts the monocyte class of white cells. Monocytes develop into phagocytic cells which consume microbial debris, but also release additional mediators of inflammation. Similarly in bone, monocytes are attracted by inflammatory signals and develop into osteoclasts which act like phagocytes to resorb existing bone. Chronic inflammation triggers the progression of both oral mucosal damage and generalized bone loss.

### 2.1.1 Oral Mucosal Disorders

Oral mucosal disorders in mammals arise in association with oral microbial growth, microbe-related mucosal tissue damage, and associated tissue inflammation. Periodontal diseases, including periodontitis and gingivitis, are caused by a complex microbial biofilm present on the surfaces of the teeth and in the gingival sulcus or pocket which forms at the junction of gum tissue and tooth surface. Periodontal disease involves inflammation of gingival tissues in response to the actions of oral microbes, primarily bacteria. Accumulation of calculus, or dental plaque, initiates and sustains oral mucosal inflammation. Dental plaque is characterized by a crystallized mass adhering to enamel surfaces of teeth, composed of a mixed colony of bacteria in a matrix of bacterial, epithelial and leucocyte cell remnants.

In oral mucosal inflammation, which accompanies gingivitis and periodontitis, the gingiva appear red and swollen, and have a tendency to bleed when the teeth are brushed. As the disease progresses, the attachment between the gums and the tooth may be broken. This creates a space, the periodontal pocket, between the tooth and gum which serves as a center for enhanced microbial growth. Such growth can lead to the formation of abscesses and bone loss in the alveolar crest. The consequence of advanced periodontal disease is a loosening of the teeth and ultimately tooth loss.

Persistent oral microbial growth and oral mucosal tissue inflammation are associated with a spectrum of pathology including calculus accumulation (dental plaque), apthous ulcers, oral malodor (halitosis), gingivitis, periodontitis, alveolar bone loss, and loss of teeth (Preshaw et al., Current concepts in periodontal pathogenesis. Dent Update. 2004; 31(10):570-2, 574-8). All aspects of oral mucosal inflammation and oral mucosal pathology are increased in tobacco users, including those who smoke and/or chew tobacco (Bergstrom, Tobacco smoking and chronic destructive periodontal disease. Odontology 2004; 92(1):1-8).

Periodontitis can affect a broad population from pre-pubertal to adult, generally increasing in prevalence and severity with increasing age. Periodontitis may also be a secondary problem in persons with other diseases such as patients receiving cancer chemotherapy, radiation therapy, or those afflicted with arthritis. In addition, the presence of active periodontitis is known to be a primary contributor to the causation and severity of important systemic diseases including atherosclerosis (Chun et al., Biological foundation for periodontitis as a potential risk factor for atherosclerosis. J Periodontal Res. 2005; 40(1):87-95), rheumatoid aritiritis (Mercado et al., Inter-relationships between rheumatoid arthritis and periodontal disease. A review. J Clin Periodontol. 2003; 30(9):761-72), pre-eclampsia during pregnancy (Canakci et al., Periodontal disease as a risk factor for pre-eclampsia: A case control study. Aust N Z J Obstet Gynaecol. 2004; 44(6):568-73), and preterm birth (Dortbudak et al., Periodontitis, a marker of risk in pregnancy for preterm birth. J Clin Periodontol. 2005; 32(1):45-52; Yeo et al., Periodontal Disease—The Emergence of a Risk for Systemic Conditions: Pre-term Low Birth Weight. Ann Acad Med Singapore. 2005; 34(1):111-6).

Current methods of treatment of periodontal diseases depend on the severity of the disease. Mild cases are generally treated by the mechanical removal of gingival and subgingival irritants such as calculus through scaling of lateral tooth surfaces. More severe cases are treated surgically by removal of gingival tissue and polishing of tooth roots. Surgical intervention is a painful and costly procedure.

Routine oral hygiene to prevent and control periodontal disease includes cleansing of teeth and gingiva using toothbrush-applied dentifrice (toothpaste and toothpowder), mechanical removal of oral debris using dental floss, and oral rinsing with mouthwashes. In periodontal disease, oral hygiene and the additional use of systemic and topical oral antibacterial agents have not been shown to be effective at reversing the progression of oral mucosal damage (Preshaw, Antibiotics in the treatment of periodontitis. Dent Update. 2004; 31(8):448-50, 453-4, 456). This includes the use of anti-microbials such as chlorhexidine, stannous fluoride, phenolic compounds, cetylpyridinium chloride and triclosan in oral rinses and toothpaste. Some of these formulations utilize quaternary ammonium compounds or benzalkonium chloride as a preservative or germicide among numerous other ingredients. Illustrations of these uses are described in U.S. Pat. Nos. 4,110,429, and 5,374,418. Although the contribution of chronic inflammation to oral mucosal disorders has been well documented, a limited number of compositions and methods of treatment have been developed based on the use of anti-inflammatory compounds in this spectrum of disorders (Scannapiec, Periodontal inflammation: from gingivitis to systemic disease? Compend Contin Educ Dent. 2004; 25(7 Suppl 1):16-25).

Prophylactic measures can be taken to forestall the occurrence, or reoccurrence of periodontal disease. Known prophylactic measures include regular removal of calculus by dental hygienists, and personal use of dental floss and gingival irrigation using hydromagnetic irrigators. Such measures are generally time consuming, expensive, involve a strict regimen of care, and are rarely sufficient in cases of severe periodontitis. Regular use of common mouthwashes and toothpastes may actually contribute to oral inflammation due to pro-inflammatory ingredients. Alcohol, typically present at 26% concentration in mouthwash, causes tissue damage and increases the inflammatory potential of other mouthwash ingredients (Muller et al., Tissue damage in the rabbit oral mucosa by acute and chronic direct toxic action of different alcohol concentrations. Exp Pathol. 1983; 24(2-3):171-81). Sodium Lauryl Sulfate (SLS), a common surfactant in toothpaste, irritates oral mucosal tissue (Baert et al., The effect of sodium lauryl sulphate and triclosan on hamster cheek pouch mucosa. Int J Exp Pathol. 1996; 77(2):73-8). Improved compositions which can be ingested as orally active agents for systemic anti-inflammatory activity and conveniently applied to the teeth and gingiva during routine oral hygiene are needed. Ideally, such compositions would intervene with both the microbial origins and associated tissue inflammation of periodontal disease.

### 2.1.1.1 The Inflammatory Response is an Important Component of Oral Mucosal Disorders

Growth of oral microbes results in the vicious cycle of epithelial cell loss providing a rich culture media for further microbial overgrowth. Porphyromonas gingivalis, a gram-negative anerobe, is an important colonizer of gingival tissues, contributing to periodontitis. The response of bystander cells in oral tissue to P. gingivalis and other microbes is to activate a cellular inflammatory response, releasing soluble mediators of inflammation, including inflammatory cytokines. Cytokines attract the influx of various white blood cells of the immune system including T-lymphocytes, monocytes, and macrophages. The presence of antigenic and inflammatory bacterial products in the oral mucosa, such as bacterial lipopolysaccharides (LPS), activate monocytes, macrophages and lymphocytes causing the release of more inflammatory mediators and cytokines. The process of chewing with inflamed gingiva causes the release and systemic circulation of many of the inflammatory factors from gingival tissue which may contribute to inflammatory processes at sites distant from the oral cavity.

The presence of strains of oral bacteria associated with severe periodontitis triggers the release of inflammatory cytokines, and is associated with elevated circulating levels of C-reactive protein (CRP), a serum marker of inflammation (Craig et al., Relationship of destructive periodontal disease to the acute-phase response. J Periodontol. 2003; 74(7):1007-16). Individuals who have inherited more reactive inflammatory cytokine profiles have been noted to also have more severe periodontal disease (Quappe et al., Association of interleukin-1 polymorphisms with aggressive periodontitis. J Periodontol. 2004; 75(11):1509-15).

The presence of active cyclooxygenase-2 (COX-2) enzyme, a contributor to inflammation, is elevated in periodontitis (Zhang et al., The overexpression of cyclo-oxygenase-2 in chronic periodontitis. J Am Dent Assoc. 2003; 134(7):861-7). The use of anti-inflammatory compounds in the form of COX-2 enzyme inhibitors has been proposed as an approach to the treatment of inflammatory disorders of the head and neck, including periodontitis. However, clinical responses to the use of selective and non-selective COX-2 inhibitors have not been shown to provide clinically significant advantages over mechanical plaque removal alone (Vardar et al., Effects of selective cyclooxygenase-2 inhibition on gingival tissue levels of prostaglandin E2 and prostaglandin F2alpha and clinical parameters of chronic periodontitis. J Periodontol. 2003; 74(1):57-63). COX-2 inhibitors also increase heart attack and stroke rates and are therefore no longer considered safe (Levesque et al., The Risk for Myocardial Infarction with Cyclooxygenase-2 Inhibitors: A Population Study of Elderly Adults. Ann Intern Med. 2005; epub). Higher dose use of COX-2 inhibitors has been reported to have paradoxical, pro-inflammatory activity activating the transcription factor Nuclear Factor Kappa Beta (NF-kappa B) which promotes inflammation (Niederberger et al., Celecoxib loses its anti-inflammatory efficacy at high doses through activation of NF-kappaB. FASEB J. 2001; 15(9):1622-4).

Recently, activation of NF-kappa B has been documented in mucosal tissue involved in periodontitis. Based on gingival tissue assays, the disease process of oral mucosal inflammation includes activation of NF-kappa B (Sabeti et al., Detection of receptor activator of NF-kappa beta ligand in apical periodontitis. J Endod. 2005; 31(1):17-8). Activated NF-kappa B has been suggested as a general cellular marker of inflammation that is associated with an overactive immune response and inflammation-related tissue damage. Activation of NF-kappa B is also associated with cancer and confers resistance to apoptosis (programmed cell death) in many types of cancer cells.

### 2.1.2 Importance of Bone-Related Disease

Millions of people worldwide are affected by chronic bone-related diseases. These include otherwise healthy women and men with age-related loss in the density and strength of bones, termed “osteopenia” (mild loss) and “osteoporosis” (severe loss). In addition, individuals with certain chronic inflammatory conditions show accelerated development of osteopenia and osteoporosis. This category of bone loss has been best described for Rheumatoid Arthritis (Mikuls T R, Saag K G, Curtis J, Bridges S L Jr, Alarcon G S, Westfall A O, Lim S S, Smith E A, Jonas B L, Moreland L W; CLEAR Investigators. Prevalence of osteoporosis and osteopenia among African Americans with early rheumatoid arthritis: the impact of ethnic-specific normative data. J Natl Med Assoc. 2005 August; 97(8):1155-60) and Systemic Lupus Erythematosus (Bultink I E, Lems W F, Kostense P J, Dijkmans B A, Voskuyl A E. Prevalence of and risk factors for low bone mineral density and vertebral fractures in patients with systemic lupus erythematosus. Arthritis Rheum. 2005 July; 52(7):2044-50). Osteoporosis is now a major public health threat. Due to its rising prevalence and to the huge cost of treating osteoporosis-associated bone fractures, better means of preventing and treating osteopenia and osteoporosis are needed.

Osteopenia and Osteoporosis are characterized by a progressive decline in bone mass and density, resulting in fragile bones and increased risk of fracture. Approximately 10 million Americans have osteoporosis, and an additional 18 million have ostopenia. Eighty percent of affected individuals are postmenopausal women, but 8 to 15 million men older than 50 years have osteoporosis or low bone density. Women experience a phase of rapid bone loss at menopause, followed by subsequent slower bone loss during the 60's, 70's, and beyond. Older men experience an exponential increase in the incidence of osteoporosis starting in their 60's. The disorder typically remains silent until a fracture occurs, most frequently at the spine, hip, or distal radius (Kirk D, Fish S A. Medical management of osteoporosis. Am J Manag Care. 2005 July; 10(7 Pt 1):445-55).

### 2.1.2.1 Osteoblasts, Osteoclasts and Estrogen Effects

Maintainance of bone health requires continued deposition of new bone matrix by osteoblasts, balanced by appropriate remodeling and resorption of bone matrix by osteoclasts. Both osteoblasts and osteoclasts are cells derived from progenitors that reside in the bone marrow; osteoblasts belong to the mesenchymal cell lineage of the marrow stroma, and osteoclasts to monocytic cells from the hematopoietic cell lineage. The development of osteoclasts from their progenitors is dependent on stromal-osteoblastic cells and is regulated by sex steroids and inflammatory cytokines. Estrogen deficiency is the main cause of bone loss in postmenopausal women and may also contribute to bone loss in aging men. Estrogen acts directly on both osteoblasts and osteoclasts through high-affinity estrogen receptors. When gonadal function is lost, the formation of osteoclasts as well as osteoblasts is altered in the bone marrow. Estrogen deficiency increases activation and decreases apoptosis of mature osteoclasts. The cellular activity of the bone marrow is further altered by the process of aging. Specifically, senescence may decrease the ability of the marrow to form osteoblast precursors. The association between the dysregulation of osteoclast or osteoblast development in the marrow and the disruption of the balance between bone resorption and bone formation, results in age-related loss of bone. Excessive osteoclastogenesis and inadequate osteoblastogenesis are considered in part responsible for the mismatch between the formation and resorption of bone in postmenopausal and age-related osteopenia. Increased osteoclastogenesis with aging is associated with higher circulating levels of inflammatory cytokines, particularly Tumor Necrosis Factor Alpha (TNF-α), Interleukin-1 (IL-1), and Interleukin-6 (IL-6).

Smoking accelerates bone loss, in part due to increased metabolism of estrogen. Moreover, cigarette smokers tend to be thinner and undergo earlier menopause. A cross-sectional study of female twins who were discordant for tobacco use found that smoking one pack of cigarettes per day throughout adulthood was associated with a 5% to 10% lower Bone Mineral Density (BMD) (Hopper J L, Seeman E. The bone density of female twins discordant for tobacco use. N Engl J Med. 1994 Feb. 10; 330(6):387-92). Other studies have demonstrated an association between cigarette smoking and a decreased BMD response to hormone replacement in postmenopausal women (Bjarnason N H, Christiansen C. The influence of thinness and smoking on bone loss and response to hormone replacement therapy in early postmenopausal women. J Clin Endocrinol Metab. 2000 February; 85(2):590-6). Consequently, smoking cessation is recommended to protect and maintain bone health and slow aging-related and inflammation-associated bone loss. All aspects of bone loss are increased in tobacco users, including those who smoke and/or chew tobacco (Benson B W, Shulman J D. Inclusion of tobacco exposure as a predictive factor for decreased bone mineral content. Nicotine Tob Res. 2005 October; 7(5):719-24).

### 2.1.2.2 Current Therapy for Osteopenia and Osteoporosis

Nondrug therapy for osteopenia and osteoporosis consists of calcium and vitamin D, exercise, and smoking cessation. These components have been examined primarily as preventive measures for age-related bone loss and fracture occurrence. Recent randomized controlled trials have indicated that supplementation with calcium and vitamin D alone are not adequate to reduce aging related fracture risk (Jackson R D, LaCroix A Z, et al., and Women's Health Initiative Investigators. Calcium plus vitamin D supplementation and the risk of fractures. N Engl J Med. 2006 Feb. 16; 354(7):669-83). While Estrogen Replacement Therapy (ERT) has proven effective in reversing age-related bone loss in post menopausal women, recent reports documenting safety isssues of increased breast cancer, heart disease, and strokes in clinical trials of ERT have sharply curtailed this mode of therapy for age-related bone loss. Current medications for age-related bone loss include both antiresorptive and anabolic types. Antiresorptive medications—estrogens, selective estrogen receptor modulators (raloxifene), steroid derivatives (tibolone), bisphosphonates (alendronate, risedronate, and ibandronate) and calcitonins—work by reducing rates of bone remodeling. Teriparatide (parathyroid hormone) is the only anabolic agent currently approved for osteoporosis in the United States (Kirk D, Fish S A. Medical management of osteoporosis. Am J Manag Care. 2004 July; 10(7 Pt 1):445-55).

### 2.2 DIM And DIM-Related Indoles are Cancer Preventive Compounds which Selectively Promote Programmed Cell Death (Apoptosis)

Cruciferous vegetables contain a family of plant protective compounds called glucosinolates which give rise to active compounds with indole rings exemplified by indole-3-carbinol (I3C). Oral ingestion of I3C results in the gastric conversion of I3C into at least twenty acid condensation products, many of which are bioavailable, the most prevalent of which include CTR (cyclic trimer; 5,6,11,12,17,18-hexahydrocyclonona[1,2-b:4,5-b′:7,8-b″]triindole), HI-IM (1-(3-hydroxymethyl)-indolyl-3-indolylmethane), DIM (diindolylmethane), ICZ (indolocarbazole) and LTr-1 (linear trimer; [2-(indol-3-ylmethyl)-indol-3-yl]indol-3-ylmethane) (Stresser et al., 1995, Drug Metabolism and Disposition 23:965-975). The fact that there are many non-DIM acid condensation products of I3C, produced in vivo at equal or greater levels as DIM, which can be responsible for I3C's activity, requires that biologic activities of individual condensation products like DIM be demonstrated directly.

As one of many products derived from I3C, DIM is also present in cruciferous plants following release of I3C. Once formed, DIM is stable in acid. In cell culture, isolated DIM has been shown to have apoptosis promoting effects in both estrogen-dependent and independent breast cancer cells (Hong et al., 2002, Biochem Pharmacol. 63:1085-97). In animals, orally administered DIM inhibits the growth of certain chemically induced forms of breast cancer (Chen et al., 1998, Carcinogenesis 19:1631-9). Similarly, synthetic DIM-related indoles have been described in U.S. Pat. Nos. 6,800,655; 6,656,963; and 5,948,808, which are also active as cancer treatments in animals. Recently, DIM has been shown to specifically induce apoptosis in Human Papilloma Virus (HPV) oncogene altered cervical cancer cell lines (Chen et al., 2001, J Nutr. 131:3294-302). This cell culture work demonstrated that DIM was more active than I3C in inducing markers of apotosis. Other non-DIM I3C condensation products were not tested. Further work has utilized DIM in the cell culture of prostate cancer cell lines demonstrating it to have anti-androgen activity similar to non-indole antiandrogen drugs (Le et al., 2003, J Biol Chem. 278:21136-45). However, in vivo studies in mice suggest that expected effective plasma levels of DIM are not easily achieved in humans (Anderton et al., 2004, Drug Metab Dispos. 32:632-8).

Regarding prior human uses of DIM, various formulations and dosage forms for systemic DIM uses have been described. These include oral formulations with enhanced absorption to promote estrogen metabolism (U.S. Pat. No. 6,086,915), oral and topical administration of DIM to treat mastalgia, endometriosis, and cervical dysplasia (U.S. Patent Application Publication No. 2004/0072891 and U.S. Pat. No. 6,689,387), and oral and topical administration of DIM to treat Human Papilloma Virus (HPV) related conditions (U.S. Patent Application Publication No. 2003/0096855). Mastalgia (breast pain) is a pain disorder not associated with histological evidence of inflammation (Watt-Boolsen et al., 1982, Fibrocystic disease and mastalgia. A histological and enzyme-histochemical study. Dan Med Bull. 29:252-4).

In addition to promoting apoptosis in breast cancer cells, DIM has recently been shown to inhibit the activation of NF-Kappa B, as evidenced by the reduced presence of NF-Kappa B protein in the nuclei of breast cancer cells after treatment with DIM (Rahman et al., 2005, Inhibition of nuclear translocation of nuclear factor-{kappa}B contributes to 3,3′-diindolylmethane-induced apoptosis in breast cancer cells. Cancer Res. 65:364-71). More recently, DIM has been shown to be an immunomodulator, increasing the production of interferon gamma in breast cancer cells (Xue et al., 2005, DIM stimulates IFNgamma gene expression in human breast cancer cells via the specific activation of JNK and p38 pathways. Oncogene, 24:2343-53).

The activities of DIM have not been studied in relation to inflammatory disorders or mucosal inflammation. Based on conflicting results of DIM activity in cell culture studies, it is difficult to predict DIM's effects in vivo on cancer, infections, or inflammation-related processes. DIM has been shown to activate the Mitogen Activated Protein Kinase (MAPK) cell signaling pathway in cell culture (Leong et al., 2004, Mol Endocrinol. 18:291-302). Activated MAPK is associated with cancer promotion, and the promotion of inflammation (Takanami-Ohnishi et al., 2002, J Biol Chem. 277:37896-903). Since DIM has been identified as a compound which activates MAPK activity (Leong et al., 2004, Mol Endocrinol. 18:291-302), and increased MAPK activity is known to result from the presence of Porphyromonas gingivalis, an important bacteria contributing to gingivitis and periodontitis (Darveau et al., 2002, Infect Immun 70:1867-73), DIM would not be expected to be of benefit in treating oral mucosal disorders. It is believed that no prior oral or topical anti-inflammatory uses of DIM have been proposed or described for any condition.

Regarding bone health, the known activities of DIM to promote 2-hydroxylation of estrogen taught in U.S. Pat. No. 6,086,915, viewed in the context of recent scientific information, would be expected to put women and men treated with DIM at greater, not less, risk for accelerated bone loss. Recent reports have shown a strong association of 2-hydroxy estrogen production with osteoporosis and osteopenia. Therefore, based on its estrogen metabolic effects, DIM would be expected to worsen bone status in women and men at risk for osteoporosis and osteopenia. This clinical data teaches that supplemental DIM use would be contra-indicated to promote bone health. Three clinical studies have demonstrated that women with higher 2-hydroxylation of estrogen have lower bone density. Individuals with the highest 2-hydroxy estrogen levels had the lowest bone mineral density in a study of post menopausal women (Lim S K, Won Y J, Lee J H, Kwon S H, Lee E J, Kim K R, Lee H C, Huh K B Chung B C. Altered hydroxylation of estrogen in patients with postmenopausal osteopenia. J Clin Endocrinol Metab. 1997 April; 82(4):1001-6), and postmenopausal women, followed prospectively, had the greatest loss of bone density if they started the study with the highest 2-hydroxyestrone to 16-hydroxyestrone urinary ratio (Leelawattana R, Ziambaras K, Roodman-Weiss J, Lyss C, Wagner D, Klug T, Armamento-Villareal R, Civitelli R. The oxidative metabolism of estradiol conditions postmenopausal bone density and bone loss. J Bone Miner Res. 2000 December; 15(12):2513-20). The well documented association of tobacco smoking with accelerated osteoporosis also teaches that DIM use in osteopenia and osteoporosis would be contra-indicated, since smoking, like DIM use, causes an increase in estrogen 2-hydroxylation (Michnovicz J J, Hershcopf R J, Naganuma H, Bradlow H L, Fishman J. Increased 2-hydroxylation of estradiol as a possible mechanism for the anti-estrogenic effect of cigarette smoking. N Engl J Med. 1986 Nov. 20; 315(21):1305-9).

### 2.3 Phytoestrogens

Phytoestrogens are plant compounds with properties similar to those of estrogens. They are available in the United States as dietary supplements and have been promoted by manufacturers for menopausal complaints, primarily hot flushes. A positive effect on bone health from phytoestrogen intake has been postulated based on the lower incidence of osteoporosis-related fractures in Asian countries, where phytoestrogens are routinely consumed. Soy isoflavones are a group of dietary phytoestrogen compounds believed to be beneficial in the treatment and/or prevention of osteoporosis. U.S. Pat. No. 5,424,331 by Shlyankevich describes a composition for the treatment or prevention of osteoporosis that utilizes soy bean food products that contain natural phytoestrogens of the isoflavone or coumestan groups. The soy isoflavones include daidzein, genistein, and glycitein. Isoflavones can also be derived synthetically.

Genistein's action of increasing the bone mineral amount has been recently clarified. Genistein has an effect of inhibiting the function of osteoclasts that cause the lysis of bone mineral which is about 10 times more potent than that of daidzein (Gao Y H, Yamaguchi M. Suppressive effect of genistein on rat bone osteoclasts: apoptosis is induced through Ca2+ signaling. Biol Pharm Bull.1999 August; 22(8):805-9). Further, it is known that genistein directly inhibits the bone resorption by osteoclasts in a culture system using a metaphysial tissue in the femora of old rats (Yamaguchi M, Gao Y H. Inhibitory effect of genistein on bone resorption in tissue culture. Biochem Pharmacol. 1998 Jan. 1; 55(1):71-6). However, in a recent clinical trial, genistein supplementation at 90 mg/day failed to reduce serum and urinary indicators of ongoing bone loss (Ibertazzi P, Steel S A, Bottazzi M. Effect of pure genistein on bone markers and hot flushes. Climacteric. 2005 December; 8(4):371-9). A year long clinical trial providing genistein at 52 mg/day similarly failed to show any measurable improvement in bone mineral density (Kreijkamp-Kaspers S, Kok L, Grobbee D E, de Haan E H, Aleman A, Lampe J W, van der Schouw Y T. Effect of soy protein containing isoflavones on cognitive function, bone mineral density, and plasma lipids in postmenopausal women: a randomized controlled trial. JAMA. 2004; 292:65-74).

A study of Ipriflavone, a synthetic isoflavone derivative, has suggested that Ipriflavone inhibits bone resorption and stimulates osteoblast activity in vitro in cell cultures and in vivo in experimental models of osteoporosis. However, when Ipriflavone was tested in a large, controlled trial at 600 mg/day, no significant improvement in measures of bone health were evident over 3 years of treatment (Alexandersen P, Toussaint A, Christiansen C, Devogelaer J P, Roux C, Fechtenbaum J, Gennari C, Reginster J Y; Ipriflavone Multicenter European Fracture Study. Ipriflavone in the treatment of postmenopausal osteoporosis: a randomized controlled trial. JAMA. 2001 Mar. 21; 285(11):1482-8). At higher doses of Ipriflavone and other phytoestrogens, concerns exist regarding safety. These concerns relate to findings of reduced lymphocyte counts and reports of uterine and mammary cell hyperproliferation.

Taken as a whole, the clinical evidence indicates that supplemental phytoestrogen use alone at the highest safe dose provides marginal efficacy at best in preventing or reversing age-related bone loss.

Therefore, more effective and consistent anti-inflammatory interventions for oral mucosal disorders and bone loss are needed. Currently marketed mouthwashes and toothpastes have anti-bacterial activity, anti-plaque activity, and breath freshening activity, but do not utilize agents specifically included for their anti-inflammatory activity. More effective treatments are needed that specifically intervene in conditions associated with oral mucosal inflammation and oral mucosal damage. New treatments are needed that address both the inflammatory and microbial aspects of the spectrum of oral mucosal pathology. Ideally, the use of improved oral health compositions will result in the improvement of objective clinical markers of tissue inflammation associated with gingivitis and periodontitis. New methods of preventing and treating bone-loss are also needed based on the aging of the population and the lack of known safe and effective nutritional and hormonal interventions for osteopenia and osteoporosis. Ideal interventions will control the inflammatory component of oral mucosal and bone loss pathophysiology and be compatible with current medical treatments.

### 3 SUMMARY OF THE INVENTION

The present invention provides methods for preventing and treating oral mucosal disorders and promoting bone health. In one embodiment, the invention provides methods for preventing or treating an oral mucosal disorder, or a symptom thereof, comprising administering to a subject in need thereof a therapeutically effective amount of Diindolylmethane (DIM), or a DIM-related indole. In a preferred embodiment, the administering is by systemic or topical oral administration.

The present invention also provides compositions for treating oral mucosal disorders comprising DIM, or a DIM-related indole, in an amount effective to reduce oral mucosal inflammation, formulated in the form of a toothpaste, oral gel, mouth wash, or chewing gum.

Oral mucosal disorders, and symptoms thereof, that can be prevented or treated by the methods and compositions of the invention include, but are not limited to, calculus accumulation (dental plaque), apthous ulcers, oral malodor (halitosis), gingivitis, periodontitis, alveolar bone loss, and loosening of teeth. In preferred embodiments, the subject is a human.

The present invention also provides methods for preventing aging-related and inflammation-associated bone loss, or a symptom thereof, comprising administering to a subject in need thereof a therapeutically effective amount of Diindolylmethane (DIM) or a DIM-related indole. In preferred embodiments, the administering is by systemic or topical administration. In preferred embodiments, the subject is a mammal, preferably a human.

In another embodiment, the present invention provides methods for preventing aging-related and inflammation-associated bone loss, or a symptom thereof, comprising administering to a subject in need thereof a therapeutically effective amount of (1) DIM or a DIM-related indole; and (2) phytoestrogen. In a preferred embodiment, the phytoestrogen is genistein. In preferred embodiments, the administering is by systemic or topical administration. The DIM, or a DIM-related indole, can be administered by a different route than the phytoestrogen. In preferred embodiments, the subject is a human.

The present invention also provides compositions for treating aging-related and inflammation-associated bone loss comprising DIM, a DIM-related indole, or DIM in combination with a selected phytoestrogen, in an amount effective to reduce indicators of bone loss, formulated in the form of a tablet, capsule, drink mix, fortified food, or chewing gum.

Aging-related and inflammation-associated bone loss, and symptoms thereof, that can be prevented or treated by the methods and compositions of the invention include but are not limited to bone fractures, osteoporotic pain syndromes, inflammatory arthritis, Rheumatoid Arthritis associated bone loss, Systemic Lupus Erythematosis associated bone loss, alveolar bone loss, and loosening of teeth. In certain embodiments of the invention bone loss is characterized by diminished bone density on x-ray bone densitometry, loss of vertical height, alveolar bone loss on physical exam, or abnormal serum or urinary bone health markers.

In a preferred embodiment, the methods and composition of the invention comprise the use of DIM. DIM-related indoles suitable for use in the methods and compositions of the invention include, but are not limited to, hydoxylated DIMs, methoxylated DIMS, 2-(Indol-3-ylmethyl)-3,3′-diindolylmethane (LTR), hydroxylated LTRs, methoxylated LTRS, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane. In a preferred embodiment, DIM or the DIM related indole is purified.

In promoting bone health and preventing or treating bone loss with DIM, or DIM-related indoles, preferred, selected phytoestrogens for use in the methods and compostions of the invention include genistein (4′,5,7-trihydroxyisoflavone), ipriflavone (3-phenyl-7-propan-2-yloxy-chromen-4-one), equol ((3S)-3-(4-hydroxyphenyl)chroman-7-ol), Red clover derived isoflavone extract (biochanin A, formononetin), and/or an isopropanolic extract from the rhizomes of Cimicifuga racemosa (Black Cohosh). In a most preferred embodiment, the phytoestrogen is genistein. In a preferred embodiment, the phytoestrogen is purified.

Systemic treatment of oral mucosal disorders and aging-related and inflammation-associated bone loss includes the ingestion of oral formulations of DIM, or a DIM-related indole, formulated for adequate gastrointestinal absorption. Ideally, DIM, or a DIM-related indole, is formulated for enhanced bioavailablity. In one embodiment, DIM, or a DIM-related indole, is processed with phosphatidyl choline. In one embodiment, DIM, or a DIM-related indole is microencapsulated with tocopheryl succinate polyethylene glycol 1000 (TPGS), preferably in a capsule or tablet. In another embodiment, the DIM, or DIM-related indole, is microencapsulated and complexed with beta cyclodextrin, hydroxypropylmethylcellulose, chitosan derivatives, beta-1,3-glucan or combinations thereof for more effective oral systemic use.

For systemic use, e.g., in the form of capsules or tablets, a preferred dose range of DIM would be 25-750 mg/day or 5-750 mg/day, preferably given once, twice, or three times a day. A more preferred dose range would be 25-200 mg/day or 50-200 mg/day, preferably given in 2 divided doses every 12 hrs. For children, the dose would preferably be 0.5-4 mg/kg/day, preferably given in 2 divided doses every 12 hrs. In certain embodiments for promotion of bone health, genistein is administered with the DIM, or DIM-related indole, preferably orally in the dosage range of 25-1,000 mg/day, preferably given once, twice, or three times a day. A preferred dose range for genistein would be 25-200 mg/day, given once daily. A more preferred dose range for genistein would be 25-150 mg/day, preferably given in 2 divided doses every 12 hrs.

Topical treatment of oral mucosal disorders includes specially formulated DIM, or DIM-related indoles, administered directly to oral mucosa in the form of toothpaste, oral gels, mouth wash, mouth rinse, lozenges, chewing gum, DIM-impregnated dental floss, and dental powders. Topical treatment of aging-related and inflammation-associated bone loss includes specially formulated DIM, or DIM-related indoles, administered directly to skin in the form of topical gels, lotions, ointments, or creams. In certain embodiments, particles of microencapsulated DIM complexed with phosphatidyl choline, TPGS, cylodextrins, hydroxypropylmethylcellulose, chitosan derivatives, and beta-1,3-glucans are included in topical formulations for topical administration.

For topical formulations, the content range of DIM is preferably from 0.1-8.0%, 0.1-3.0% by weight, more preferably 0.1-2.0% by weight, of the formulation. A most preferred concentration is 0.5-2.0%. Using a DIM content of 2%, the typical dose of DIM using 1-3 grams of, for example, a toothpaste or oral gel, would be 20-60 mg applied to teeth/gums, preferably twice a day or 20-60 mg applied to skin, preferably twice a day. With a 1% DIM content each gram of a toothpaste, topical gel or cream provides 10 mg of DIM. A preferred topical dose is 20-30 mg from 2-3 grams of toothpaste, cream or gel applied once or twice a day, more preferably, 20-30 mg applied twice a day. In formulations comprising genistein, the content range of genistein is preferably from 0.1-3.0% by weight, more preferably 0.1-2.0% by weight, of the formulation. A most preferred concentration is 0.5-2.0%. Using a genistein content of 2%, the typical dose of genistein would be 20-60 mg applied to skin, preferably twice a day. With a 1% genistein content each gram of topical gel or cream provides 10 mg of genistein. A preferred topical dose is 20-30 mg from 2-3 grams of cream or oral gel applied once or twice a day, more preferably, 20-30 mg applied twice a day.

Formulations for bone health use standard approaches known in the art for manufacturing capsules and tablets. The DIM, or a DIM-related indole, together with selected phytoestrogen and optional anti-inflammatory agent can also be added to selected foods as fortified, “functional” foods. These typically include drink mixes, meal replacement powders, food bars, and candies. Formulations include DIM, or a DIM-related indole, and one or more of the following phytoestrogens compounds: genistein, ipriflavone, equol, mixed phytoestrgens from soy isoflavone extracts, and mixed phytoestrogens from red clover isoflavone extracts.

In the methods of the invention comprising administering DIM, or the DIM-related indole, in combination with a phytoestrogen, DIM, or the DIM-related indole, may be administered simultaneously or within a short time of the phytoestrogen, for example, 30 seconds, 1 minute, 5 minutes, 15 minutes, 30 minutes, 1 hour, 4 hours, 8 hours, 12 hours or 24 hours of one another.

In certain embodiments, the methods and compositions further comprise the use of an anti-inflammatory agent in conjunction with DIM-related indole with or without a phytoestrogen. In a preferred embodiment, the anti-inflammatory agent is resveratrol, an extract of Polygonium cuspidatum, silibinin, an extract of Silybum marianum, curcumin, an extract of Curcuma domestica, apigenin, aloe extract, terpenes, particularly the sequiterpene lactone, Parthenolide, citrus extracts, particularly the citrus flavonoid, nobiletin, boswellic acid, caffeic acid, and propolix extracts containing caffeic acid phenthyl ester, deguelin, extracted from various plant sources including Munduelea sericea, Evodiamine, ursolic acid, Allyl Disulfide, Andrographolide, Dehydro-Andrographolide, Deoxy-Andrographolide, Brassinin, Caffeic acid, Capsanthin, Capsaincin, L-Carnitine, L-Carnitine HCl, Carnosic acid, Chelerythrine Chloride, Cromolyn sodium, Diallyl disulfide, Diallyl sulfide, Diallyl trisulfide, Dibenzoylmethane, Ebulin 1, Ellagic acid, (−)Epicatechin, (−)Epicatechin gallate, (−)Epigallocatechin, Epigallocatechin gallate, Ferulic acid, 18β-Glycyrrhetinic Acid, Glycyrrhizic acid ammonium salt trihydrate, a Green tea polyphenol, Honokiol, 5-Hydroxy-L-tryptophan, Hypericin, Hypocrellin A, Ibuprofen, Idebenone, luteolin, D-Limonene, Limonin, Limonin Glucoside, DL-α-Lipoic acid, Melatonin, Perillyl Alcohol, Phenylbutyrate, Phenylethyl 3-mehtylcaffeate, Phenylethyl 4-methylcaffeate, Phenyl isothiocyanate, Phytic Acid, 9-cis-Retinoic acid, 13-cis-Retinoic acid, trans-Retinoic acid, all-trans-Retinol, retinyl acetate, Retinyl palmitate, Rosmarinic acid, Rutaecarpine, sulforaphane, L-Theanine, Trichostatin A, Xylitol, and/or Vitamin K3. In the methods of the invention comprising administering an anti-inflammatory, DIM, or a DIM-related indole, may be administered simultaneously or within a short time of the anti-inflammatory agent, for example, 30 seconds, 1 minute, 5 minutes, 15 minutes, 30 minutes, 1 hour, 4 hours, 8 hours, 12 hours or 24 hours of one another.

In certain embodiments, the methods and compositions of the invention can be used in combination with hormonal replacement and other established therapies for age-related bone loss.

The DIM, or DIM-related indoles, used in oral topical formulations in combination with other anti-inflammatory agents are believed to provide additive and synergistic anti-inflammatory activity which results in effective, self-administered, treatments for oral mucosal disorders. When concurrent gingival disease (gingivitis and periodontitis) is a contributing factor, the methods and compositions of the invention also provide an intervention useful for the prevention of pre-eclampsia and premature birth in pregnant women and for the treatment of rheumatoid arthritis and atherosclerosis.

The DIM, or DIM-related indoles, used in combination with selected phytoestrogens are believed to provide additive and synergistic activity for maintaining healthy bones. This allows for a lower, potentially safer, dose for DIM and selected phytoestrogen when used in combination. The methods and compositions of the present invention are used to prevent and reverse aging-associated bone loss (osteopenia and osteoporosis). The methods and compositions of the invention can also be used as a treatment for bone loss associated with chronic inflammatory conditions. In particular, the methods and compositions of the present invention can be used to prevent and treat bone loss associated with Rheumatoid Arthritis (RA) and Systemic Lupus Erythematosis (SLE).

### 4 DETAILED DESCRIPTION OF PRESENT INVENTION

The present invention provides methods and compositions for the prevention and treatment of oral mucosal disorders comprising Diindolylmethane (DIM), or a DIM-related indole. In addition, the present invention provides methods and compositions for the prevention and treatment of aging-related and inflammation-associated bone loss using Diindolylmethane (DIM), or a DIM-related indole, preferably with a phytoestrogen, e.g., genistein.

With regard to oral health, treatment of oral mucosal disorders in accordance with the present invention refers to the inhibition of, or at least the reduction of, oral mucosal inflammation associated with the disorder. “Oral mucosal disorders” are meant to include, but are not limited to, inflammatory disorders of the gingival mucosa (gums) associated with calculus accumulation (dental plaque), apthous ulcers, oral malodor (halitosis), gingivitis, periodontitis, alveolar bone loss, and loosening of teeth.

The methods of use of DIM-based treatments for oral mucosal disorders are believed to result in improvement of clinical signs of reduced oral mucosal inflammation. This includes reduction of oral inflammatory markers including plaque index, gingival bleeding, reduced gingival pocket depth, and reduced gingival redness and swelling as evidenced by normalization of standardized, oral mucosal examinations. Assessment of the efficacy of the methods and compositions of the invention in reducing these oral inflammatory markers can be measured by methods known to one of skill in the art. See, e.g., Caton et al., Associations between bleeding and visual signs of interdental gingival inflammation. J Periodontol. 1988; 59(11):722-7; and Loe, The Gingival Index, the Plaque Index and the Retention Index Systems. J Periodontol. 1967; 38(6):Suppl:610-6. Optimally, in subjects with oral mucosal disorders, DIM-based treatments result in reduced levels of local and systemic markers of inflammation including serum C-reactive protein, and serum and tissue activation markers of NF-kappa B. Assessment of the efficacy of the methods and compositions of the invention in reducing these markers can be measured using standard assays known to one of skill in the art. See, e.g., Ledue et al., Preanalytic and analytic sources of variations in C-reactive protein measurement: implications for cardiovascular disease risk assessment. Clin Chem. 2003 August ; 49(8):1258-71.

Administration of DIM, and DIM-related indoles, to subjects with oral mucosal disorders also provides intervention useful for the prevention and treatment of disorders directly tied to oral inflammation. These conditions include pre-eclampsia and premature birth in pregnant women and rheumatoid arthritis and atherosclerosis when concurrent gingival disease (gingivitis and periodontitis) is a contributing factor.

With regard to the promotion of bone health, the present invention provides methods and compositions for the prevention and treatment of aging-related and inflammation-associated bone loss using Diindolylmethane (DIM), or a DIM-related indole. The present invention also provides methods and compositions for the prevention and treatment of aging-related and inflammation-associated bone loss using (1) DIM or a DIM-related indole; and (2) genistein. In this regard, treatment of aging-related and inflammation-associated bone loss in accordance with the present invention refers to the inhibition of, or at least the reduction of, symptoms or laboratory indicators of aging-related and inflammation-associated bone loss. “Aging-related and inflammation-associated bone loss” is meant to include, but is not limited to, osteopenia, osteoporosis, elevated serum or urinary markers of bone loss, Rheumatoid Arthritis associated bone loss, Systemic Lupus Erythematosis associated bone loss, alveolar bone loss, and loosening of teeth.

The DIM-based treatments for aging-related and inflammation-associated bone loss are believed to result in improvement of clinical signs of accelerated bone loss. This includes reduction in serum or urinary markers of bone loss, reduction in frequency of fractures, particularly hip fractures, and reduction in pain associated with osteoporosis. Assessment of the efficacy of the methods and compositions of the invention in reducing evidence of aging-related and inflammation-associated bone loss can be measured by methods known to one of skill in the art (See, e.g., Herrmann M, Kraenzlin M, Pape G, Sand-Hill M, Herrmann W. Relation between homocysteine and biochemical bone turnover markers and bone mineral density in peri- and post-menopausal women. Clin Chem Lab Med. 2005; 43(10):1118-23). The most widely used method for diagnosing osteoporosis is bone mineral density (BMD) measurement. Dual-energy x-ray absorptiometry scanning is the most appropriate technique for measuring bone density, as it yields precise measurements at clinically important sites (spine and hip) with minimal radiation exposure. The World Health Organization (WHO) defines osteoporosis as a BMD of at least 2.5 SDs below the mean for young adults of the same race and sex (T score), and osteopenia as a T score of 1.0 to 2.5 SDs below the mean.

Optimally, in subjects with aging-related and inflammation-associated bone loss, DIM-based treatments result in reduced levels of local and systemic indicators of bone loss and activation markers of NF-kappa B. Assessment of the efficacy of the methods and compositions of the invention in reducing these markers can be measured using standard assays known to one of skill in the art. See, e.g., Faure P, Ramon O, Favier A, Halimi S. Selenium supplementation decreases nuclear factor-kappa B activity in peripheral blood mononuclear cells from type 2 diabetic patients. Eur J Clin Invest. 2004 July; 34(7):475-81.

Administration of DIM, and DIM-related indoles, or the combination of DIM with phytoestrogens, including genistein, to subjects with aging-related and inflammation-associated bone loss also provides intervention useful for the prevention and treatment of certain systemic inflammatory disorders. These conditions include Rheumatoid Arthritis (RA) and Systemic Lupus Erythematosis (SLE).

Formulations suitable for use in the methods and compositions of the invention for oral mucosal health use standard approaches for manufacturing toothpastes, oral gels, and mouthwash formulations. Mouthwash formulations are preferably formulated with limitation on the alcohol content. In preferred embodiments, the alcohol content is no more than 25%, preferably no more than 15%, more preferably in the range of 5-15%. Formulations include DIM, DIM-related indoles, and one or more of the following: anti-inflammatory agents, antibacterial agents, anti-fungal agents, calcium salts, nutrients, pentetration enhancers, stabilizers, preservatives, and flavors. Preferable as additives to formulations for oral topical use are citrus extracts containing anti-inflammatory flavones. An exemplary extract is prepared from Citrus depressa and contains the polymethoxyflavonoid, nobiletin. Other useful anti-inflammatory extracts include those prepared from Commiphora mukul, containing guggulsterone, those prepared from Simon Sweet Potato, containing caffeic acid phenethyl ether (CAPE), and those prepared from Tanacetum parthenium, containing parthenolide.

Formulations suitable for use in the methods and compositions of the invention for bone health use standard approaches for manufacturing capsules, tablets, topical gels, creams, suppositories, transdermal patches, chewing gum, and fortified foods. Formulations include DIM (or a DIM-related indole), and one or more of the following phytoestrogens: genistein (4′,5,7-trihydroxyisoflavone), ipriflavone (3-phenyl-7-propan-2-yloxy-chromen-4-one), equol ((3S)-3-(4-hydroxyphenyl)chroman-7-ol), Red clover derived isoflavone extract (biochanin A, formononetin), and/or and/or an isopropanolic extract from the rhizomes of Cimicifuga racemosa (Black Cohosh). In a most preferred embodiment, the formulation comprises DIM and genistein.

In certain embodiments, the methods and compositions further comprise the use of an anti-inflammatory agent in conjunction with DIM-related indole and, optionally, a phytoestrogen. In a preferred embodiment, the anti-inflammatory agent is resveratrol, an extract of Polygonium cuspidatum, silibinin, an extract of Silybum marianum, curcumin, an extract of Curcuma domestica, apigenin, aloe extract, terpenes, particularly the sequiterpene lactone, Parthenolide, citrus extracts, particularly the citrus flavonoid, nobiletin, boswellic acid, caffeic acid, and propolix extracts containing caffeic acid phenthyl ester, deguelin, extracted from various plant sources including Munduelea sericea, Evodiamine, ursolic acid, Allyl Disulfide, Andrographolide, Dehydro-Andrographolide, Deoxy-Andrographolide, Brassinin, Caffeic acid, Capsanthin, Capsaincin, L-Carnitine, L-Carnitine HCl, Carnosic acid, Chelerythrine Chloride, Cromolyn sodium, Diallyl disulfide, Diallyl sulfide, Diallyl trisulfide, Dibenzoylmethane, Ebulin 1, Ellagic acid, (−)Epicatechin, (−)Epicatechin gallate, (−)Epigallocatechin, Epigallocatechin gallate, Ferulic acid, 18β-Glycyrrhetinic Acid, Glycyrrhizic acid ammonium salt trihydrate, a Green tea polyphenol, Honokiol, 5-Hydroxy-L-tryptophan, Hypericin, Hypocrellin A, Ibuprofen, Idebenone, luteolin, D-Limonene, Limonin, Limonin Glucoside, DL-α-Lipoic acid, Melatonin, Perillyl Alcohol, Phenylbutyrate, Phenylethyl 3-mehtylcaffeate, Phenylethyl 4-methylcaffeate, Phenyl isothiocyanate, Phytic Acid, 9-cis-Retinoic acid, 13-cis-Retinoic acid, trans-Retinoic acid, all-trans-Retinol, retinyl acetate, Retinyl palmitate, Rosmarinic acid, Rutaecarpine, sulforaphane, L-Theanine, Trichostatin A, Xylitol, and/or Vitamin K3. In the methods of the invention comprising administering an anti-inflammatory agent, DIM, or the DIM-related indole, may be administered simultaneously or within a short time of the anti-inflammatory agent, for example, 30 seconds, 1 minute, 5 minutes, 15 minutes, 30 minutes, 1 hour, 4 hours, 8 hours, 12 hours or 24 hours of one another.

### 4.1 Diindolylmethane-Related Indoles

The DIM-related indoles or DIM compounds useful in the methods and compositions of the invention include DIM (3,3′-diindolylmethane), the related linear DIM trimer (2-(indol-3-ylmethyl)-3,3′-diindolylmethane [also written: 2 (Indol-3-ylmethyl)-indol-3-yl]indol-3-ylmethane] (LTR), the DIM precursor Indole-3-carbinol [also written: 3-Indolemethanol], and the indole glucosinolate, glucobrassicin, found in cruciferous vegetables. As used herein, “DIM-related compound”, “DIM-related indole”, and “DIM derivative” are used interchangeably, and refer to both natural metabolites and analogs of DIM, and also to “structurally-related, synthetically-derived, substituted diindolylmethane compounds” and “synthetic derivatives of DIM”, such as those disclosed herein and known in the art. As used herein, “cruciferous-related indoles” encompasses the terms “DIM-related compound”, “DIM-related indole”, and “DIM derivative”. One of ordinary skill in the art will recognize that in any of the pharmaceutical compositions or methods of the invention where DIM is used, a DIM-related compound, including a structurally-related, synthetically-derived, substituted diindolylmethane compound or synthetic derivative of DIM, can be used.

The chemical structure of a DIM is as follows (where each of the R groups is H):

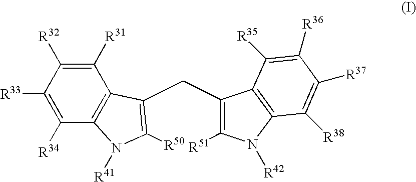

The chemical structure of LTR is as follows (where each of the R groups is H):

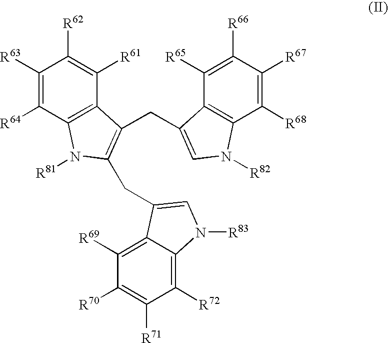

In certain embodiments, an active hydroxylated or methyoxylated metabolite of DIM, i.e., a compound of formula I, wherein R³², R³³, R³⁶, and R³⁷ are substituents independently selected from the group consisting of hydrogen, hydroxyl, and methoxy, and R³¹, R³⁴, R³⁵, R³⁸, R⁴¹, R⁴², R⁵⁰, and R⁵¹ are hydrogen, is utilized.

In certain embodiments, an active hydroxylated or methyoxylated metabolite of LTR, i.e., a compound of formula II, wherein R⁶², R⁶³, R⁶⁶, R⁶⁷, R⁷⁰, and R₇₁ are substituents independently selected from the group consisting of hydrogen, hydroxyl, and methoxy, and R⁶¹, R⁶⁴, R⁶⁵, R⁶⁸, R⁶⁹, R⁷², R⁸¹, R⁸², and R⁸³ are hydrogen, is utilized.

In an alternative embodiment, active DIM derivatives with R₃₂ and R₃₆ substituents made up of ethoxycarbonyl groups, and R₅₀, R₅₁ are either hydrogen or methyl, are utilized. In another embodiment, active substituted DIM derivatives including methylated and chlorinated compounds, exemplified by those that include 5,5′-dimethylDIM (5-Me-DIM), 2,2′-dimethylDIM (2-Me-DIM), and 5,5′-dichloroDIM (5-Cl-DIM) are described in U.S. Patent Application Publication No. 20020115708 by Safe, published Aug. 22, 2002, incorporated herein by reference in its entirety, are utilized in the present invention. In another embodiment, active DIM derivatives include imidazolelyl-3,3′-diindolylmethane, including nitro substituted imidazolelyl-3,3′-diindolylmethanes, and additional DIM-related compounds described in U.S. Patent Application Publication No. 2004/0043965 by Jong, Ling, published Mar. 4, 2004, incorporated herein by reference in its entirety, are utilized.

In certain embodiments, a DIM related compound has formula (III):

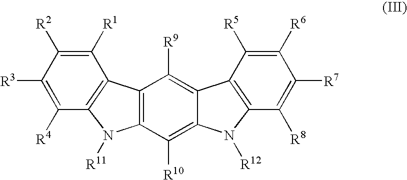

wherein:

R,¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms; and

R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,

with the provisos that: at least one of R¹, R², R³, R⁴, R⁵, R⁶, R⁷, R⁸, R⁹, R¹⁰, R¹¹ and R¹² is other than hydrogen; and when R¹, R², R³, R⁴, R⁵, R⁶, R⁷, and R⁸ are selected from hydrogen, halo, alkyl and alkoxy, then R¹¹ and R¹² are other than hydrogen and alkyl.

A preferred embodiment includes the use of 2,10-dicarbethoxy-6-methoxy-5,7-dihydro-indolo-[2,3-b]carbazole (SRI13668 (SRI Inc., Menlo Park, Calif.)). Additional preferred embodiments include the use of 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole (SRI Inc., Menlo Park, Calif.).

In another embodiment, a DIM related compound has formula (IV):

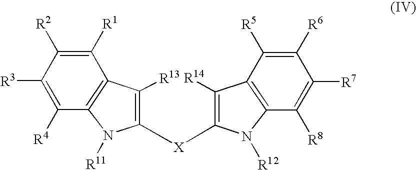

wherein:

R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;

R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl;

R¹³ and R¹⁴ are defined as for R¹, R², R³, R⁴, R⁵, R⁶, R⁷, and R⁸, with the proviso that at least one of R¹³ and R¹⁴ is other than hydrogen; and

X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹².

A preferred embodiment includes the use of 2,6-dicarbethoxy-3,3′-dimethyl-13,14-diindolylmethane (SRI Inc., Menlo Park, Calif.).

In another embodiment, a DIM related compounds has formula (V):

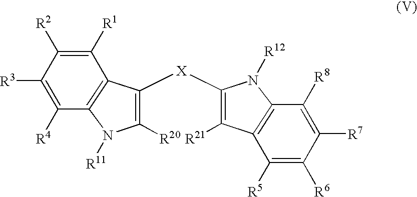

wherein:

R¹, R², R³, R⁴, R⁵, R⁶, R⁷, R⁸, R¹¹, R¹², and X are defined as for compounds of formula (III); and

R²⁰ and R²¹ are defined as for R¹, R², R³, R⁴, R⁵, R⁶, R⁷, and R⁸.

In yet another embodiment, the DIM-related indole is an indole-3-carbinol tetrameric derivative (Brandi et al., 2003, Cancer Res. 63:4028-4036).

### 4.2 Additive, Bone Health Promoting Phytoestrogens for Use with DIM and DIM-Related Indoles

A number of plant derived phytoestrogens are useful for combined use with DIM, or DIM-related indole, in the methods and compositions of the present invention for the prevention and/or treatment of aging-related and inflammation-associated bone loss. Prefered phytoestrogens for use in combination with DIM, or DIM-related indole, are genistein (4′,5,7-trihydroxyisoflavone), ipriflavone (3-phenyl-7-propan-2-yloxy-chromen-4-one), equol ((3S)-3-(4-hydroxyphenyl)chroman-7-ol), or Red clover derived isoflavone extract (biochanin A, formononetin). Most preferred is genistein. It is understood in the art that a phytoestrogen compound may have both estrogenic and anti-inflammatory activities.

### 4.3 Methods of Treatment and Prevention

### 4.3.1 Methods of Treatment and Prevention of Oral Mucosal Disorders Using DIM

Subjects with oral mucosal disorders in need of treatment present with the characteristic sign of bleeding of gums following routine brushing of teeth. Mild disease is painless, while more severe gingivititis and periodontitis are accompanied by painful gums which are swollen and red in color. On examination of gums and teeth, the severity of the oral mucosal disorder can be categorized as mild, moderate or severe based on the presence of objective signs (Loe, The Gingival Index, the Plaque Index and the Retention Index Systems. J Periodontol. 1967; 38(6):Suppl:610-6). Mild inflammation of gum tissue is associated with slight change in color to red from pink in areas of the gums, little change in texture of the gingival tissue, slight bleeding on probing of the periodontal sulcus, and probing depths of 2-3 mm. Moderate gum disease is often termed “gingivitis” and is associated with change in color from pink to red involving the entire gingival surface, moderate glazing, slight swelling, bleeding with flow on probing, and probing depths of 4-5 mm. Severe gum disease, termed “periodontitis”, is characterized by distinct redness of gum tissue, overgrowth of tissue (hypertrophy), tense swelling of gum tissue, spontaneous bleeding or ulceration of gum tissue, copious and persistent bleeding following probing, and probe depths of 5-6 mm with evidence of wide “pocket” formation. Pocket formation is due to enlargement of the periodontal sulcus surrounding the subgingival portion of the teeth. Severe periodontitis is also associated with evidence of alveolar bone loss on physical exam with probing and on x-ray examination of the jaw. Calculus accumulation may be apparent in association with mild, moderate, or severe mucosal inflammation. Oral malodor or “halitosis” often accompanies moderate and severe oral mucosal inflammation.

The methods of treatment described in the following sections are exemplary. Other methods of treatment will be apparent from the methods and compositions described herein.

### 4.3.1.1 Treatment of Mild Oral Mucosal Inflammation

Following routine dental hygiene with removal of dental calculus, subjects with mild gingival inflammation benefit by routine, twice a day application with a toothbrush of a dentrifrice (toothpaste or toothpowder) containing DIM, or a DIM-related indole, in a concentration of 0.25-1.0% by weight of the formulation. Flossing of the teeth following brushing helps to move particles of DIM from the toothpaste into the periodontal sulci for better contact with the effective tissue. Optionally, a mouthwash containing DIM, or a DIM-related indole, optionally complexed with cyclodextrins, in a concentration of 0.25-1.0% by weight of the formulation is used, preferably before sleep.

Alternatively, a gel dentifrice is used which contains DIM, or a DIM-related indole, in a concentration of 0.5-2.0% by weight of the formulation, and one or more of silybinin in a concentration of 0.25-2.0% by weight of the formulation, resveratrol in a concentration of 0.25-2.0% by weight of the formulation, tea tree oil in a concentration of 0.25-1.0% by weight of the formulation, and optionally other NF-kappa B inhibitors.

### 4.3.1.2 Treatment of Moderate Oral Mucosal Inflammation

Following routine dental hygiene with removal of dental calculus, subjects with moderate oral mucosal inflammation benefit from the combined use of systemic DIM, or a DIM-related indole, regular use of a DIM, or a DIM-related indole, containing dentifrice, and application of an oral gel containing DIM, or a DIM-related indole, after brushing teeth. A typical oral preparation for systemic administration contains 150 mg of processed DIM (37.5-50 mg/capsule of DIM), taken with water twice daily. Alternatively, the oral preparation contains 150 mg of processed DIM (37.5-50 mg/capsule of DIM) combined with silibinin (200 mg of SiliPhos, Idena, Inc.) in a single capsule, taken with water twice daily. In addition to the oral preparation, subjects with moderate oral mucosal inflammation practice oral hygiene with twice daily application by toothbrush of a dentifrice (toothpaste or toothpowder) containing DIM, or a DIM-related indole, in a concentration of 0.25-2.0% by weight of the formulation. Following brushing, a toothbrush is used to apply an oral gel containing DIM in a concentration of 0.5-1.5% by weight of the formulation. The oral gel is applied along the tooth-gum margin with a toothbrush following brushing and before flossing of teeth.

Alternatively in place of the DIM toothpaste, a gel dentifrice containing DIM, or a DIM-related indole, in a concentration of 0.5-3.0% by weight of the formulation also contains one or more of the following: silybinin in a concentration of 0.25-2.0% by weight of the formulation, resveratrol in a concentration of 0.25-2.0% by weight of the formulation, tea tree oil in a concentration of 0.25-1.0% by weight of the formulation, and other, NF-kappa B inhibitors.

### 4.3.1.3 Treatment of Severe Oral Mucosal Inflammation

Subjects with severe oral mucosal inflammation require irrigation of periodontal sulci and root planning to remove sub-gingival calculus and debris in addition to routine dental hygiene. Following dental hygienic care, subjects with severe mucosal inflammation benefit with the combined use of systemic DIM, or a DIM-related indole, regular use of a DIM, or a DIM-related indole, containing dentifrice, and application of an oral gel containing DIM, or a DIM-related indole, after brushing teeth. Additionally, once daily sulcular irrigation using a pulsating oral irrigator may be initiated. For subjects with severe oral mucosal disease, a typical oral regimen for systemic administration includes two capsules containing 150 mg of processed DIM (37.5-50 mg/capsule of DIM) taken with water twice daily. Alternatively, two capsules of an oral preparation which contains 150 mg of processed DIM (37.5-50 mg/capsule of DIM) combined with silibinin (200 mg of SiliPhos, Idena, Inc.) in a single capsule may be taken with water twice daily. In addition to the oral preparation, subjects with severe oral mucosal inflammation practice oral hygiene with twice a daily application by toothbrush of a dentrifrice (toothpaste or toothpowder) containing DIM, or a DIM-related indole, in a concentration of 0.25-3.0% by weight of the formulation. Following brushing, a toothbrush is used to apply an oral gel containing DIM, or a DIM-related indole, in a concentration of 0.5-3.0% by weight of the formulation. Alternatively, a gel dentifrice containing DIM, or a DIM-related indole, in a concentration of 0.5-3.0% by weight of the formulation also contains one or more of the following: silybinin in a concentration of 0.25-2.0% by weight of the formulation, resveratrol in a concentration of 0.25-2.0% by weight of the formulation, tea tree oil in a concentration of 0.25-1.0% by weight of the formulation, and other NF-kappa B inhibitors. The oral gel is typically applied along the tooth-gum margin with a toothbrush following brushing.

In addition to the above, once daily sulcular irrigation may be performed by the subject before the application of the oral gel. This is accomplished using a Hydro Floss® hydromagnetic irrigator following the manufacture instructions (Hydro Floss Inc., Bessemer, Ala.). An irrigation solution which contains DIM, or a DIM-related indole, in a concentration of 0.25-1.5% by weight of the formulation is utilized. Preferably, the irrigation solution contains DIM, or a DIM-related indole, in a concentration of 0.25-1.0% by weight of the formulation complexed with beta cyclodextrins in combination with tea tree oil and optionally, other additional NF-kappa B inhibitors.

Alternatively, for severe periodontitis, DIM, or a DIM-related indole, in a concentration of 0.5-3.0% by weight of the formulation, can be incorporated in long lasting polymeric gels which are introduced into diseased periodontal sulci by a dentist. The use of such solidifiying gels, which may also contain anti-inflammatory agents described herein, are described in U.S. Pat. No. 5,368,859, “Biodegradable system for regenerating the periodontium”.

### 4.3.1.4 Treatment of Non-Oral Mucosal Disorders Related to Oral Inflammation

Administration of DIM, and DIM-related indoles, to subjects with oral mucosal disorders also provides intervention useful for the prevention and treatment of disorders directly tied to oral inflammation. These conditions include pre-eclampsia and premature birth in pregnant women and rheumatoid arthritis and atherosclerosis when concurrent gingival disease (gingivitis and periodontitis) is a contributing factor. See, e.g., Canakci et al., Periodontal disease as a risk factor for pre-eclampsia: A case control study. Aust N Z J Obstet Gynaecol. 2004; 44(6):568-73; Yeo et al., Periodontal Disease—The Emergence of a Risk for Systemic Conditions: Pre-term Low Birth Weight. Ann Acad Med Singapore. 2005; 34(1):111-6; Mercado et al., Inter-relationships between rheumatoid arthritis and periodontal disease. A review. J Clin Periodontol. 2003; 30(9):761-72; and Chun et al., Biological foundation for periodontitis as a potential risk factor for atherosclerosis. J Periodontal Res. 2005; 40(1):87-95.

The group of oral mucosal disorders and secondary inflammatory disorders (including, but not limited to, rheumatoid arthritis (RA), systemic lupus erythematosous (SLE), and atherosclerosis) are effectively treated, or are believed to be effectively treated, by the systemic ingestion and oral application of compositions containing DIM and/or DIM-related indoles. The DIM, or DIM-related indole, is optionally used in combination with selected additional anti-inflammatory agents which contribute to the DIM-related therapeutic effect. Preferably, the additional anti-inflammatory agent provides overlapping inhibition of the NF-kappa B cell signaling pathway. The DIM, or DIM-related indole, may also be optionally used in combination with selected additional antibacterial agents.

Treatment of these related conditions follows those methods described for the treatment of moderate and severe mucosal inflammation.

### 4.3.1.5 Methods of Prophylaxis for Oral Mucosal Disorders

Methods are provided for the prevention of oral mucosal disorders. Administration of DIM, or a DIM-related indole, can be used as part of routine dental hygiene. Methods of prevention will typically be similar to methods for treatment of mild oral mucosal inflammation. In cases of pre-existing immune deficiency or anticipated oral mucosal inflammation which follows radiation or chemotherapy for cancer, treatment of oral mucosal disorders according to the present invention is initiated in advance of the signs and symptoms of periodontal disease. This includes the systemic and oral topical uses of DIM, or DIM-related indole, used alone or in conjunction with NF-kappa B inhibitor agents.

### 4.3.2 Methods of Treatment and Prevention of Disorders Associated with Bone Loss Using DIM

Subjects with aging-related and inflammation-associated bone loss in need of treatment present with the characteristic evidence of diminished bone density on x-ray bone densitometry, loss of vertical height, alveolar bone loss on physical exam. Early aging-related and inflammation-associated bone loss is asymptomatic, while more severe bone loss is accompanied by bone fractures, alveolar bone loss, loss of teeth, inflammatory arthritis, RA, and SLE. All forms of aging-related and inflammation-associated bone loss are accelerated and worsened by tobacco exposure.

The methods of treatment described in the following sections are exemplary. Other methods of treatment will be apparent from the methods and compositions described herein.

### 4.3.2.1 Methods of Prophylaxis to Prevent Bone Loss

Methods are provided for the prevention of aging-related and inflammation-associated bone loss. Administration of DIM, or a DIM-related indole, can be used as part of a regime of preventive nutritional supplementation alone or in combination with a selected phytoestrogen. In a preferred embodiment, the phytoestrogen is genistein. In cases of positive family history for osteopenia or osteoporosis pre-existing anticipated bone loss in at-risk family members, use of DIM, with or without phytoestrogen, according to the present invention is initiated in advance of the signs and symptoms of bone loss. This includes the systemic and oral topical uses of DIM, or DIM-related indole, used alone or in conjunction with selected phytoestrogen and optional additional NF-kappa B inhibitor agents.

### 4.3.2.2 Methods of Treatment to Promote Bone Health and Prevent Bone Loss

Methods are provided for the treatment of aging-related and inflammation-associated bone loss. Administration of DIM, or a DIM-related indole, can be used as part of a regime of therapeutic nutritional supplementation alone or in combination with a selected phytoestrogen. In a preferred embodiment, the phytoestrogen is genistein. This includes the systemic and oral topical uses of DIM, or DIM-related indole, used alone or in conjunction with selected phytoestrogen and optional additional NF-kappa B inhibitor agents.

### 4.3.2.3 Treatment of Bone Loss Associated with Chronic Inflammatory Conditions

Administration of DIM, and DIM-related indoles, preferably in combination with a phytoestrogen, such as genistein, to subjects provides intervention useful for the prevention and treatment of disorders directly tied to inflammation-associated bone loss. These conditions include Rheumatoid Arthritis (RA) and Systemic Lupus Eryethematosis (SLE). The bone loss and associated inflammation with RA and SLE are effectively treated, or are believed to be effectively treated, by the systemic ingestion and/or topical application of compositions containing DIM, or a DIM-related indoles. The DIM, or DIM-related indole, is optionally used in combination with selected additional anti-inflammatory phytoestrogens which contribute to the DIM-related therapeutic effect. In a preferred embodiment, the phytoestrogen is genistein. Preferably, the additional anti-inflammatory phytoestrogen agent provides overlapping inhibition of the NF-kappa B cell signaling pathway.

### 4.4 Administration and Dosage

In the context of oral mucosal disorders, the term “therapeutically effective amount” means an amount of DIM, or DIM-related indole, sufficient to prevent or treat oral mucosal disorders without causing intolerable side effects. In the context of bone health, the term “therapeutically effective amount” means an amount of DIM, or DIM-related indole, sufficient to prevent, slow or reverse aging or disease-related decline in bone mineral density and/or bone strength. The term “therapeutically effective amount” in the context of the combination of (1) DIM, or DIM-related indole, and (2) a phytoestrogen, such as genisterin, means that the combination is sufficient to prevent, slow or reverse aging or disease-related decline in bone mineral density and/or bone strength. The DIM and/or phytoestrogen may be present in the combination in amounts that are not therapeutically effective when adminstered individually. A treatment effect in a population of subjects may be detected by a change in an indicator of bone health indicating bone preservation. Relevant indicators of bone health include, bone densitometry, measurement of bone mineral density at lumbar spine (BMD-LS) and total hip (BMD-HIP), and fasting venous blood and urine sampling for serum Osteocalcin (OC), serum calcium (Ca), urinary desoxypyridinoline cross-links (DPD), serum osteoprotegerin (OPG) and serum soluble receptor activator of NF-kappaB ligand (sRANKL). In certain embodiments, DIM, or the DIM-related indole, is in the form of a simple particulate, which include crystalline DIM. In preferred embodiments, DIM, or a DIM-related indole, is in the form of an absorbable particulate.

Precise dosages of the compositions, e.g., DIM, optionally with a phytoestrogen, appropriate for use to treat an individual are established in appropriately controlled clinical trials and case studies. As will be appreciated, the appropriate dosage of the present composition will vary with the administrable form of the composition. It will be appreciated that the amounts of DIM, or other DIM-related indole, and, optionally, an anti-inflammatory, anti-bacterial agent, or a bone-health promoting phytoestrogen, e.g., genistein, required for said treatments will vary according to the route of administration, the oral mucosal disorder to be treated, age, and medical history of the subject, the galenic formulation of the pharmaceutical composition, etc. A subject in which an oral mucosal disorder or bone mineral loss, osteopenia, or osteoporosis is intended to be prevented or treated using the methods of the invention is preferably, a mammal, e.g., a dog, cat, horse. In a most preferred embodiment, the subject is a human.

In one embodiment, for use in treating individuals with oral mucosal disorders or individuals with risk of or existing Osteopenia or osteoporosis, a therapeutically effective amount of the compositions described herein is administered systemically. Typically, this is accomplished by administering to an individual in need a therapeutically effective amount, typically 5-750 mg/day or 25-750 mg/day, preferably 25-300 mg per day, of DIM, or a DIM-related indole, in a formulation contained in capsules or tablets that is absorbable in the gastrointestinal tract. For systemic use in the form of capsules or tablets, the dose range of DIM would be 5-750 mg/day or 25-750 mg/day given once or twice a day. A preferred dose range would be 50-200 mg/day given in 2 divided doses every 12 hrs. For children, generally with oral mucosal disorders, the dose would typicaly be 0.5-4 mg/kg/day given in 2 divided doses every 12 hrs. In certain embodiments, an absorbable formulation of DIM, or a DIM-related indole, is combined in capsules with an absorbable particulate formulation of anti-inflammatory agent, such as Silibinin, Boswellic acids, EGCG, and/or Resveratrol.

For bone health, a particulate formulation of DIM, or a DIM-related indole, is combined in capsules, tablets, drink mixes, or food bars with an particulate formulation of phytoestrogen, such as genistein or equol. In certain embodiment, the particulate formulation is absorbable. Optionally, vitamin D, with or without particulate formulations of Calcium and Magnesium salts, is added to the capsules containing absorbable DIM and absorbable phytoestrogen. Preferred phytoestrogen formulations for the present invention include, isolated genistein, isolated equol, and ipriflavone. Most preferred in genistein.

In another embodiment, a therapeutically effective amount of a composition described herein is administered topically. For topical delivery of DIM, or DIM-related indole, to the oral cavity of an individual for a suitable period of time, oral gels, toothpastes, or mouth washes are preferred formulations. For topical delivery of DIM, or DIM-related indole, to the skin of an individual for a suitable period of time, topical gels, creams, and lotions are preferred. For topical formulations, the content range of DIM is from 0.1-8.0%, 0.1-3.0% by weight, more preferably 0.1-2.0% by weight, of the formulation. A preferred concentration is 0.5-2.0%. Using a DIM content of 2%, the typical dose of DIM using 1-3 grams of a topical formulation, e.g., a toothpaste or oral gel, would be 20-60 mg or 5-60 mg applied to teeth/gums twice a day. With a 1% DIM content each gram of the topical formulation, e.g., a toothpaste or oral gel, provides 10 mg of DIM. A preferred topical dose is 20-30 mg from 2-3 grams of toothpaste or oral gel applied once or twice a day, more preferably, 20-30 mg applied twice a day.

For example, it is anticipated that an effective treatment regimen will involve the administration of a dosage in the range of at least about 10 mg DIM/g up to about 40 mg DIM/g of paste or gel, or up to an amount of DIM, or DIM-related indole, which is capable of being absorbed by the paste or gel while still being cost effective. In gum formulations, a range of at least about 1 mg DIM/stick of gum up to about 300 mg DIM/stick of gum, or up to an amount of DIM, or DIM-related indole, which is capable of being absorbed by the gum carriers while still being cost effective; and a range of about 0.001-5 g DIM/liter of liquid rinse or spray, a preferred dosage of which being represented by about 0.04 g of DIM, or DIM-related indole/liter.

In order to be effective in treating oral mucosal disorders topically, the present compositions must be in contact with affected areas of the oral cavity for an acceptable period of time per use, and must be used at least 1 to 3 times daily. The treatment time will vary with the administrable form of the composition. For example, in the form of a rinse, the composition is preferably used for 30-60 seconds per use, 2-3 times daily; in the form of a gel or paste, the composition is preferably used for about 1-2 minutes, 2-3 times daily; and in the form of a gum, the composition can be used for a longer period of time than other dosage forms, generally for at least several minutes per stick of gum.

In prefered embodiments, the methods and compositions for bone health further comprise the combined use of DIM, or DIM-related indole, in combination with selected phytoestrogens known to be useful in promoting bone health. Preferred, selected phytoestrogens include genistein (4′,5,7-trihydroxyisoflavone), ipriflavone (3-phenyl-7-propan-2-yloxy-chromen-4-one), equol ((3S)-3-(4-hydroxyphenyl)chroman-7-ol), or Red clover derived isoflavone extract (biochanin A, formononetin). Genistein is administered with the DIM, or DIM-related indole preferably orally in the dosage range of 25-1,000 mg/day, preferably given once, twice, or three times a day. A preferred dose range for genistein would be 25-200 mg/day, given once daily. A more preferred dose range for genistein would be 25-150 mg/day, preferably given in 2 divided doses every 12 hrs. For children, the dose would preferably be 0.5-4 mg/kg/day, preferably given in 2 divided doses every 12 hrs. For topical formulations, the content range of genestein is preferably from 0.1-3.0% by weight, more preferably 0.1-2.0% by weight, of the formulation. A most preferred concentration is 0.5-2.0%. Using a genistein content of 2%, the typical dose of genistein would be 20-60 mg applied to skin, preferably twice a day. With a 1% genistein content each gram of topical gel or cream provides 10 mg of genistein. A preferred topical dose is 20-30 mg from 2-3 grams of cream or oral gel applied once or twice a day, more preferably, 20-30 mg applied twice a day.

Equol would be used in the same dosage range as genistein. Ipriflavone would be used at double the dose range for genistein.

Other routes of administration which may be suitable for the methods and compositions of the invention, particularly intravenous administration, are described in U.S. Provisional Patent Application No. 60/640,301, incorporated by reference herein in its entirety.

### 4.5 Nutritional and Pharmaceutical Compositions

The present invention provides formulations for both systemic administration and topical application, for the oral cavity or the skin, for the prevention and treatment of oral mucosal disorders and/or for the promotion of bone health and prevention and treatment of osteopenia and osteoporosis. Formulations for systemic administration include DIM, or a DIM-related indole, formulated for adequate gastrointestinal absorption from a capsule or tablet. In certain embodiments, DIM, or a DIM-related indole, can be administered with additional anti-inflammatory agents, such as silibinin or boswellic acids, and/or antibacterial agents, formulated for gastrointestinal absorption and administered in capsules or tablets. In certain embodiments, DIM, or a DIM-related indole, can be administered with additional phytoestrogen agents, such as genistein, ipriflavone, or equol, formulated for gastrointestinal absorption and administered in capsules or tablets. Formulations are for human and veterinary use.

The compositions of the present invention may be consumed by mouth as pills or applied orally in an amount effective to promote oral hygiene, to reduce or prevent periodontal disease, and to alleviate inflammatory diseases of the oral cavity. The preferred compositions of the present invention include capsules, tablets, toothpastes, oral gels, mouthwashes, mouth rinses, lozenges, chewing gum, impregnated dental floss, and dental powders.

Use of additional microbicidals, solubility enhancers and mucosal penetration enhancers further improves topical formulations. The addition of pharmaceutically acceptable cyclodextrins, methycellulose derivatives, e.g., hydroxypropylmethylcellulose, essential oils, including limonene, perrillyl alcohol, tea tree oil and terpene derivatives, helps to dissolve the DIM-related indoles and improve mucosal penetration. Preferred solubility/pentration enhancers are limonene, tea tree oil, beta-cyclodextrins, and chitosan derivatives. In a preferred embodiment, the formulation of DIM-containing oral treatment products use approaches to manufacture of toothpastes, oral gels, and mouthwash formulations which include cyclodextrins, methylcellulose derivatives, chitosan derivatives, and beta-1,3-glucans. DIM and other poorly soluble ingredients are alternatively formed into sustained-release particles using methylcellulose derivatives, chitosan derivatives, and beta-1,3-glucans which can be suspended in oral gels or pastes for better adherence and higher mucosal penetration following application to oral mucosa.

Oral topical formulations of DIM, or DIM-related indole, include formulations of toothpaste, oral gels, mouthwashes, mouth rinses, lozenges, chewing gums, DIM-impregnated dental floss, and dental powders. Suitable processes for incorporating DIM, or DIM-related indoles, in dental floss products are described in U.S. Pat. Nos. 5,209,251 and 5,765,576. Ideally, the DIM, or DIM-related indole, is formulated in a gel dentifrice with visco-elastic and bioadhesive properties that allow the formulation to flow into periodontal sulci and have particles of DIM, with or without other active compounds, retained in contact with the oral mucosa. Preferably, the dentifrice will be hydrophilic, become increasingly flowable when present in the oral cavity at body temperature, contain polymeric bioadhesive ingredients which will sequester particles of DIM and, optionally, other anti-inflammatory compounds and/or antibacterial compounds, and resist clearance of active compounds due to the flow of saliva.

Topical formulations of DIM, or DIM-related indole, for the skin include formulations of gels, creams, ointments, lotions, and sustained release patches. Ideally, the DIM, or DIM-related indole, is formulated in a topical gel, cream, and lotion with adequate penetration enhancing activity that allow the active ingredients to penetrate and circulate in the blood of the treated subject.

In certain embodiments, DIM, or the DIM-related indole, is in the form of a particulate, which includes crystalline or unprocessed DIM (such as DIM available from Designed Nutritional Products, West Orem, Utah). In preferred embodiments, DIM, or a DIM-related indole, is in the form of an absorbable particulate. Preferably, the DIM, or DIM-related indole, used in the invention has been processed to enhance bioavailability, for example, in an absorption enhancing formulation at 25-500 mg per day as a suspension of microparticles in a starch carrier matrix, as is described in U.S. Pat. No. 6,086,915, incorporated herein by reference in its entirety; however any suitable preparation of pure diindolylmethane can be used in the methods and compositions of the invention.

Structurally-related, synthetically-derived, substituted diindolylmethane's, as described by Jong (U.S. Patent Application Publication No. 2004/0043965) are administered according to the present invention in an acceptable formulation for oral administration in a dose of 10-400 mg/day. Preferably, these substituted diindolylmethanes are administered in an absorption-enhanced formulation at a dose of 50 to 250 mg/day. For systemic administration of Diindolylmethane (DIM) or a DIM-related indole, advantageous formulation methods include the use of cyclodextrins, methylcellulose derivatives, and chitosan derivatives, which are provided in the following sections.

In other embodiments, the DIM particle is mixed with a second or third active, anti-inflammatory ingredient such as Silibinin or Resveratrol. As an example, capsules containing the combination of 150 mg of processed Diindolylmethane and 10-30 mg of Resveratrol (from 300 mg of Regrape X) (Interpharma Praha, CZ), are made by mixing the processed Diindolylmethane, Regrape X, with microcrystaline cellulose or rice flour excipient and placing the mixed powder into opaque gelatin capsules. As an alternative example, capsules containing the combination of 150 mg of processed Diindolylmethane and 100-300 mg of and absorbable formulation of silibin (Silybin) [Silybin from Siliphos®, [Indena, Inc., #IdB 1016] are made by mixing the processed Diindolylmethane, SiliPhos, with microcrystaline cellulose or rice flour excipient and placing the mixed powder into opaque gelatin capsules. A preferred combination contains 150 mg of processed Diindolylmethane and 200 mg of SiliPhos per capsule.

In other embodiments, the DIM particle is mixed with a second or third active, phytoestrogen ingredient such as genistein, ipriflavone, or equol. As an example, capsules containing the combination of 100 mg of processed Diindolylmethane and 50-200 mg of genistein (from Bonistein™, DSM Nutritional Products ) are made by mixing the processed Diindolylmethane and Bonistein, with microcrystaline cellulose or rice flour excipient and placing the mixed powder into opaque gelatin capsules. As an alternative example, capsules containing the combination of 100 mg of processed Diindolylmethane and 50-150 mg of an absorbable formulation of Ipriflavone [Ostivone, Cheisi Farmaceutici SpA (Parma, Italy)] are made by mixing the processed Diindolylmethane, Ipriflavone, with microcrystaline cellulose or rice flour excipient and placing the mixed powder into opaque gelatin capsules. A preferred combination contains 100 mg of processed Diindolylmethane and 150 mg of Ipriflavone per capsule. Additional, preferred anti-inflammatory inhibitors of NF-kappa B for promoting oral mucosal and bone health are listed in Table 1.

### 4.5.1 Exemplary Formulations

The following include detailed exemplary descriptions of compositions according to the present invention.

I. Oral DIM tablets/capsules using particles formulated for enhanced absorption

Nutritional and Pharmaceutical Dosage Forms for DIM-related indoles used in treating oral mucosal disorders or promoting bone health and preventing and/or treating osteopenia and osteoporosis: Multi-application DIM-related indole containing particles are manufactured by various techniques including spray drying, spray cooling, selective precipitation, crystallization and other particle forming methods. The resulting particles are used in the manufacture of the following dosage forms, some of which are described in U.S. Pat. No. 6,086,915, incorporated by reference herein in its entirety.

II. Spray Dried Microencapsulated solid dispersions
1. TPGS/phosphospholipid spray-dried particles. Production of absorption-enhanced DIM-related indole particle formation is provided in U.S. Pat. No. 6,086,915.
2. Liquid emulsions using TPGS/phosphospholipid spray-dried particles. Production of emulsions for oral use utilizes absorption-enhanced DIM-related indole particle formation is provided in U.S. Pat. No. 6,086,915.
3. Flavored DIM granules for oral use (Chocolate, Orange “sprinkles”). Production of flavored granules for oral use utilizes absorption-enhanced DIM-related indole particles (DIM/TPGS) as provided in U.S. Pat. No. 6,086,915. Production steps include dry mixing DIM/TPGS particles with maltodextrin granules, addition of flavoring particles and granulation using a standard fluid bed granulator.

III. Spray Dried Polymer Based Solid Dispersions

Production techniques for DIM-related indoles may utilize those described in U.S. Patent Application Publication No. 20030072801, entitled “Pharmaceutical compositions comprising drug and concentration-enhancing polymers,” herein incorporated by reference in its entirety. In particular, production involves the following dissolution regulating polymers, used with and without lipid stabilizers:
1. Polymer included: Hydroxy Propyl Methylcellulose
2. Polymer: Hydroxy Propyl Cellulose

IV. Cyclodextrin Based Formulations

Examples of manufacturing techniques are described in U.S. Pat. No. 4,877,778 and U.S. Patent Applications Publication Nos.: 20040053888; 20030073665; and 20020068720, each of which is herein incorporated by reference in its entirety. Using cyclodextrin loading production techniques to incorporate DIM-related indoles, the following final formulations are produced:
1. Dry particle complex for oral use
2. Dry particle complex for inclusion as a component of oral gels, toothpastes, toothpowders, and chewing gum.

### 4.5.2 Oral-Topical DIM-Based Formulations for Treatment of Oral Mucosal Disorders

The oral topical compositions according to the present invention preferably comprise one or more pharmaceutically acceptable carriers and the active constituents, e.g., a DIM-related indole and optionally, an anti-inflammatory agent and/or antibacterial agent. In utilizing the formulations of the present invention, topical treatments of oral mucosal disorders according to the present invention are benefited by exposing inflamed oral mucosa to higher concentrations of DIM-related indoles for more prolonged periods of time. Preferred methods include formulation of DIM, with or without an anti-inflammatory agent and/or an antibacterial agent, in slowly dissolving, sustained-release, particles which increase the exposure of oral mucosa to the active agents. Additional, desirable characteristics to optimize the concentration of active ingredient in the tissue of oral mucosa for sustained release particles of DIM and anti-inflammatory agents include bioadhesive, penetration enhancing, and anti-bacterial activity. The most desirable materials to be utilized to form the matrix of particles containing active ingredients used in manufacture of topical formulations will therefore also possess additional activities. Bioadhesive and penetration enhancement further increases that contact of the particle with the oral mucosa and improves penetration of active agents into the mucosa. Calcium salts are preferably included as a carrier to promote topical therapeutic activity of DIM and DIM-related indoles. The delivery of the active agents of the present invention is best accomplished by forming small particles of the active compounds that are then suspended in the oral gel, toothpaste, or mouthwash. Specialized carriers compatible with oral topical formulations are used.

The preferred materials with bioadhesive activity include methylcellulose and modified methyl cellulose derivates, chitosan and modified chitosan derivatives, and glucans, particularly beta-1,3,-glucans. Chitosan derivatives provide bioadhesive, penetration enhancing activity and antibacterial activity (Sandri et al., Assessment of chitosan derivatives as buccal and vaginal penetration enhancers. Eur J Pharm Sci. 2004; 21(2-3):351-9). Beta-1,3-glucans, particularly soluble beta-1,3-glucans, possess bioadhesive and immune stimulating activity related to the stimulation of immune and mucosal dendritic cells (de Felippe et al., Infection prevention in patients with severe multiple trauma with the immunomodulator beta 1-3 polyglucose (glucan). Surg Gynecol Obstet. 1993; 177(4):383-8). Therefore, Chitosan derivatives and Beta-1,3-glucan concentrates are preferred to be utilized in conjunction with methylcellose polymers to form the particle matrix for spray-dried DIM material to be used in manufacture of oral gels, toothpaste, toothpowder, mouthwash, and chewing gum.

Calcium salts are preferred as additives to toothpaste, toothpowder, oral gels, mouthwashes, chewing gum, and to matrix forming material for DIM containing particles, since the presence of dissolved calcium contributes to DIM activity (Xue et al., DIM stimulates IFNgamma gene expression in human breast cancer cells via the specific activation of JNK and p38 pathways. Oncogene, 2005 Mar. 31; 24(14):2343-53.). Preferred calcium salts are soluble in aqueous environments such as saliva and include Calcium Chloride (CaCl₂), Calcium Floride (CaFl₂), Calcium Citrate, Calcium Hydroxide, Calcium Lactate, Calcium Phosphate, and Calcium Sulfate. The prefered concentration range of calcium salts as percent weight of final formulations is 0.02-2.0%. In a preferred embodiment, CaCl₂, CaFl₂, and Calcium Citrate (Calcium Citrate Tribasic Tetrahydrate) are each present at 0.1% of formula weight to give a total calcium salt content of 0.3%.

Manufacturing of DIM-containing particles is accomplished according to formulation methods which involve co-dissolving DIM, release-controlling polymers, bioadhesives, and penetration enhancers and spray drying the mixture to form particles. This process produces complex particles of appropriate micron and sub-micron size which adhere to oral mucosa and enter periodontal sulci. Preferably, particles are formed using methycellulose derivatives (Hydroxypropylmethylcellulose [HPMC]), other methycellulose derivatives, cyclodextrins (Hydroxypropyl b-cyclodextrin [HPCD]), soluble glucans (Beta-1,3-glucan [BetaPrecise-909 and BetaPrecise-929, Cypress Systems, Inc., Fresno, Calif.]), and chitosan derivatives, including N-carboxymethylchitosan, and Methyl-pyrrolidinone chitosan (Sandri et al., Assessment of chitosan derivatives as buccal and vaginal penetration enhancers. Eur J Pharm Sci. 2004; 21:351-9). Other useful particle forming agents with bioadhesive qualities are Poly(vinyl pyrrolidone), Sodium carboxymethyl cellulose, Hydroxy ethyl cellulose, Poly(vinyl alcohol), Poly(isobutylene), and Poly(isoprene). The HPMC/HPCD DIM particles are preferred for use in manufacture of oral gels, toothpastes, toothpowders, mouthwashes, and chewing gums of the present invention. Manufacturing techniques providing examples for the incorporation of beta-1,3-glucans derived from curdlan and Brewer's Yeast (Saccharomyces cerevisiae) into formulations, such as hydrogels, useful in the present invention are provided in U.S. Pat. No. 5,346,935, herein incorporated by reference in its entirety. Techniques of utilization of chitosan derivatives in combination with other viscogenic agents suitable for topical oral mucoal formulations are described in U.S. Patent Application Publication No. 20020142041. Techniques of incorporating chitosan in controlled release formulations for oral care are described in U.S. Patent Application Publication No. 20030152629.

Other particle matrix forming materials optionally used to incorporate in the oral gels, toothpastes, mouthwashes and chewing gums of the present invention include aloe extracts, hydroxypropyl cellulose, hydroxypropyl methylcellulose, hydroxyethylcellulose, ethylcellulose, carboxymethyl cellulose, dextran, gaur-gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, polyhyaluronic acid, casein, gelatin, glutin, polyanhydride, polyacrylic acid, alginate, poly(methyl methacrylate), poly(ethyl methacrylate), poly(butyl methacrylate), poly(isobutyl methacrylate), poly(hexl methacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate), poly(fumaric-co-sebacic)acid and mixtures thereof.

The methods for producing mixed composition particles incorporating DIM-related indoles and other NF-kappa B inhibitors typically include the steps of: (i) heating an appropriate solvent and dissolving or dispersing the DIM-related indole and optionally, an anti-inflammatory agent and/or antibacterial agent, to form a melt; (ii) dissolving or dispersing the polymeric matrix material and bioadhesive material in a separate aqueous phase to form a composition; (iii) heating the composition to a temperature above of the mixture formed in step (i); (iv) mixing said hot melt of step (i) with the aqueous solution formed in step (ii) to form a dispersion; (v) high shear homogenization of the dispersion at a temperature sufficient to maintain a stable homogenous dispersion and (vi) spray drying of the heated dispersion to form a dry powder composition of particles.

In oral topical formulations, DIM is utilized as a single active ingredient or in conjunction with other anti-inflammatory compounds with or without additional anti-bacterial compounds for improved compositions of toothpastes, toothpowders, oral topical gels, mouthwash/rinses, and chewing gums according to the present invention.

Optimal oral topical formulations of the present invention combine pleasant taste, pleasant aftertaste, pleasant smell and mouth sensation, provided by flavorings, and humectants. Ideally, the active DIM-related indoles, anti-inflammatory agents, anti-microbials, immune activators, and bioadhesives of the formulations are shelf stable and lack local or systemic toxicity when applied to oral mucosa with daily, chronic use. Individual ingredients are chosen based on safety data, cost, and compatibility with production methods, solvents used, and lack of chemical reactivity with other ingredients.

Combination formulations for oral topical uses utilizing DIM in treatment of mild, moderate and severe oral mucosal disorders are within the scope of the present invention. Such formulations are designed to optimize the oral topical activity of DIM by combining DIM, or a DIM-related indole, with complementary anti-inflammatory, antibacterial, immunomodulating, penetration enhancing and bioadhesive compounds.

Illustrations of the composition of other combined formulations utilizing DIM are provided in the following table.

TABLE 1
Summary Chart Illustrating Active Ingredients in DIM-based
Compositions for Oral Topical and Systemic Use:

| | Percent | | | | |
|---|---|---|---|---|---|
| | Wt/Wt of | | | | |
| | Topical | Oral Gel | Toothpaste | Mouthwash | |
| Active Agent | Formulations | Formulation | Formulation | Formulation | Reference |
| DIM-related indole | | | | | |
| DIM | 0.1-3.0 | A, B | C, D | E, F | |
| NF-kappa B | | | | | |
| Inhibitor | | | | | |
| DIM | 0.1-2.0 | A, B | C, D | E, F | Rahman et al., 2005, Cancer |
| | | | | | Res. 65: 364-71 |
| Silymarin | 0.1-3.0 | B | C, D | F | Manna et al., 1999, J Immunol. |
| (Silybinin) | | | | | 163: 6800-9 |
| Curcumin | 0.1-3.0 | | D | F | Aggarwal et al., 2004, Int J |
| | | | | | Cancer. 111: 679-92 |
| Boswellic acid | 0.1-3.0 | | | | Syrovets et al., 2005, J |
| | | | | | Immunol. 174: 498-506 |
| Resveratrol | 0.1-3.0 | B | C, D | F | Kundu et al., 2004, Mutat Res. |
| | | | | | 555: 65-80 |
| ECGC | 0.1-3.0 | | D | F | Jeong et al., 2004, Pharm Res. |
| (Green Tea Extract) | | | | | 21: 661-70 |
| Caffeic Acid | 0.1-3.0 | B | C, D | F | Islam et al., 2002, Biosci |
| (Simon Sweet | | | | | Biotechnol Biochem. 66: 2483-6. |
| Potato Extract) | | | | | |
| Parthenolide | 0.1-3.0 | B | C, D | F | Jain et al., 1999, J |
| Feverfew Extract | | | | | Ethnopharmacol. 68: 251-9 |
| (Tanacetum | | | | | |
| parthenium) | | | | | |
| Sambucus Extract | 0.1-3.0 | B | C, D | F | Ebrahimzadeh et al., |
| (Sambucus Niger | | | | | Fitoterapia., 2006, 77: 146-8 |
| and Elder) | | | | | |
| Deguelin | 0.1-2.0 | B | C, D | F | Chen et al., 2006, Acta |
| (Mundulea sericea) | | | | | Pharmacol Sin. 27: 485-90) |
| Guggulsterone | 0.1-3.0 | B | C, D | F | Shishodia et al., 2004, J Biol |
| (Commiphora | | | | | Chem. 279: 47148-58 |
| mukul) | | | | | |
| Antibacterial | | | | | |
| Agent | | | | | |
| ECGC | 0.1-3.0 | B | | F | Hirasawa et al., 2002, J |
| (Green Tea Extract) | | | | | Periodontal Res. 37: 433-8 |
| Tea Tree Oil | 0.01-0.2 | B | C, D | F | Groppo et al., 2002, Int Dent J. |
| | | | | | 52: 433-7 |
| Perilla | 0.1-2.0 | | D | | Yamamoto et al., 2002, Biosci |
| (Seed Extract) | | | | | Biotechnol Biochem. 66: 921-4 |
| Chitosan | 0.5-5.0 | | C, D | | Rabea et al., 2003, |
| | | | | | Biomacromolecules. 4: 1457-65 |
| Honokiol (Houpo) | 0.1-2.0 | | | F | Ho et al., 2001, Phytother Res. |
| | | | | | 15: 139-41 |
| Nutrient | | | | | |
| Zinc Salt | 0.01-1.0 | | | | |
| Vitamin C | 0.01-1.0 | | | | |
| Immunomodulator | | | | | |
| DIM | 0.1-2.0 | | | | Xue et al., 2005, Oncogene. |
| | | | | | epub 2005 Feb 07 |
| Beta-1,3-Glucan | 0.01-3.0 | | | | de Felippe et al., 1993, Surg |
| | | | | | Gynecol Obstet. 177: 383-8 |
| Penetration | | | | | |
| Enhancer | | | | | |
| Limonene | 0.01-1.5 | A, B | C, D | E, F | Al-Saidan et al., 2004, Skin |
| | | | | | Pharmacol Physiol. 17: 310-20 |
| Chitosan | 0.5-5.0 | | C, D | | Sandri et al., 2004, Eur J Pharm |
| | | | | | Sci. 21: 351-9 |
| Solvent | | | | | |
| Ethanol | 2.0-20.0 | A, B | C, D | E, F | |

A: Illustration of active ingredients used in the Oral Topical Gel with DIM in Example 7.
B: Illustration of active ingredients used in the Oral Topical Gel with DIM in Example 8.
C: Illustration of active ingredients used in the Toothpaste with DIM in as used in the Clinical Trial in Example 10.
D: Illustration of active ingredients used in a Toothpaste with DIM possible for use as a commercial product.
E: Illustration of active ingredients used in the Mouthwash with DIM in Example 7.
F: Illustration of active ingredients used in a Mouthwash with DIM possible for use as a commercial product.

### 4.5.2.1 DIM Formulated for Oral Topical Administration Directly to Oral Mucosa in the Form of Tooth Paste

In the case of a oral paste, DIM, or DIM-related indole, alone or combined with other anti-inflammatory agents and/or anti-bacterial agents, is mixed with carriers conventionally used to formulate a paste. Carriers include thickening agents such as methylcellulose or hydroxypropyl methylcellulose, abrasive agents, humectants and surfactants and cyclodextrins, penetration enhancers, particularly limonene or tea tree oil, cariostatic agents, flavoring agents, whitening agents, preservatives, coloring agents, and buffers. Due to the poor water solubility of DIM and certain NF-kappa B inhibitors, such as resveratrol, silibinin, curcumin, and evodiamine, complexation with cyclodextrins, preferably Beta-cyclodextrin, can be undertaken during the processing of individual ingredients or during mixing of ingredients. Formulation of DIM in toothpastes includes the use of cyclodextrin in oral care products as described in U.S. Patent Application No. 2004/0076591 A1, and U.S. Pat. Nos. 6,261,540; 6,534,042; 5,716,601; 5,281,410, each of which is incorporated by reference herein in its entirety.

DIM, or a DIM-related indole, may be formulated in sustained release particles with bioadhesives, cyclodextrins, and polymers. Particles are suspended in hydrogels and pastes for application to oral mucosa.

Toothpastes are formulated according to standard industry practices including the following preferred process. Carboxymethyl cellulose (weight percent 2) is dispersed in glycerin (weight percent 15) using a HOCKMEYER HVI mixer, (Hockmeyer Equipment Co., Elizabeth City, N.C.). Water (weight percent 17.5), and sorbitol (weight percent 28) are added and mixed for 25 minutes. 1% DIM weight percent of final gel (using microcrystalline DIM complexed with hydroxypropyl-beta-cyclodextrin-weight percent 10 and additional hydroxypropyl-beta-cyclodextrin (weight percent 5) are then added and mixed for a further 10 minutes. The phenolics are mixed together, i.e., tea tree oil (weight percent 0.05), eucalyptol (weight percent 0.5), and menthol (weight percent 0.5), to make a phenolic phase. The phenolic phase is added to the cellulose/sorbitol/cyclodextrin/water phase until the phenolics are dissolved. Sylodent 700 (weight percent 14), Sylox (weight percent 5), Calcium Citrate (weight percent 1), Green Tea Extract (weight percent 1) are then added and mixed thoroughly for 30 minutes. The resulting light green gel is deaerated to remove air bubbles. The product is transferred to dispensing tubes.

One example of a combination of DIM and other active anti-inflammatory agents to form a gel toothpaste includes: DIM (0.5-1.5% formula wt), glycerin (10-20% formula wt), sorbitol (15-30% formula wt), hydrated silica (6-15% formula wt), Aloe Vera gel (2-5% formula wt), Sodium Lauroyl Sarcosinate (0.01-0.5% formula wt), peppermint oil (0.01-0.5% formula wt), menthol (0.01-0.5% formula wt), Tea Tree oil (0.01-0.5% formula wt), Xylitol (0.1-2% formula wt), Green Tea extract (0.1-1% formula wt), Perilla Seed extract (0.1-1% formula wt), Stevia extract (0.1-1% formula wt), Beta-1,3-glucan (BetaPrecise-909, Cypress Systems, Inc., Calif.) (0.1-1% formula wt), Ester-C® Topical Liquid (0.1-1% formula wt), Calcium Citrate (0.1-1% formula wt), and water. Ester-C® Topical Liquid (Alcer Corp., Foothill Ranch, Calif.) is a source of Calcium, Zinc Ascorbate, and L-Ascorbic acid.

### 4.5.2.2 DIM Formulated for Oral Topical Administration Directly to Oral Mucosa in the Form of Tooth Powder

The compositions of the invention may be formulated in the form of a tooth powder. Representative methods are described in U.S. Patent Application Publication No. 2004/0076591 A1 “Cyclodextrins in Dental Products, and U.S. Pat. No. 6,261,540, “Cyclodextrins and hydrogen peroxide in dental products”; U.S. Pat. No. 6,534,042, “Taste masking of phenolics using citrus flavors”; U.S. Pat. No. 5,716,601, “Oral compositions, such as oral gels and toothpastes, containing a novel abrasive.”; and U.S. Pat. No. 5,281,410 “Methods of reducing plaque and gingivitis with reduced staining”. In an alternative method, formulations use particle formation steps as described in U.S. Patent Application Publication No. 20030152629.

### 4.5.2.3 DIM Formulated for Oral Topical Administration Directly to Oral Mucosa in the Form of Oral Topical Gel

In the case of a gel, DIM, or DIM-related indole, alone or combined with an anti-inflammatory agent and/or an anti-bacterial agents, may be admixed with gel carriers such as gelatin, polyethylene glycol, guar gum or combinations thereof. Oral gels may also include one or more of the following: antifungal agents, stabilizers, and penetration enhancers. Structural compounds are also normally present in a gel, examples of which include polyoxyethylene-polyoxypropylenecopolymers. Such structurants are generally present in amounts ranging from about 18 to about 25% by weight of the composition. Due to the poor water solubility of DIM and certain NF-kappa B inhibitors such as resveratrol, silibinin, curcumin, and evodiamine, complexation with cyclodextrins, preferably Beta-cyclodextrins, chitosan, chitosan derivatives including methyl-pyrrolidinone chitosan, and glucans, preferably beta-1,3-glucans, can be undertaken during the manufacture of oral gels. Examples of manufacturing steps using cyclodextrins in oral care products not containing DIM or NF-kappa B inhibitors are described in U.S. Patent Application Publication No. 2004/0076591 A1, and U.S. Pat. Nos. 6,261,540; 6,534,042; 5,716,601; and 5,281,410, each of which is incorporated by reference herein in its entirety. Alternative methods of formulation use particle formation steps described in U.S. Patent Application Publication Nos. 20030152629 and 20040191281.

In one method, steps for manufacture of a DIM-containing topical gel include the following. A DIM oral gel is formulated by dispersing carboxymethyl cellulose (weight percent 12) in glycerin (weight percent 15) using a HOCKMEYER HVI Mixer (Hockmeyer Equipment Co., Elizabeth City, N.C.). Water (weight percent 27.5) and sorbitol (weight percent 27), and Calcium Citrate (weight percent 1) were added and mixed for 25 minutes. 1% DIM weight percent of final gel (using microcrystalline DIM complexed with hydroxypropyl-beta-cyclodextrin-weight percent 10) and additional hydroxypropyl-beta-cyclodextrin (weight percent 5) were then added and mixed for a further 10 minutes. The phenolics were mixed together, i.e., tea tree oil (weight percent 0.5), eucalyptol (weight percent 0.5), and menthol (weight percent 0.5), to make a phenolic phase. The phenolic phase was added to the cellulose/sorbitol/cyclodextrin/water phase until the phenolics are dissolved. Green Tea Extract (weight percent 1) is then added and mixed thoroughly for 30 minutes. The resulting light green gel was deaerated to remove air bubbles. Optionally, extracts containing curcumin, silybin, and resveratrol providing these substances at 1% weight of the total formulation are added. Other ingredients in the form of essential oils, including limonene, and NF-kappa B inhibitors including Evodiamine and Honokiol, each present at 0.01-0.5% of the total formulation weight are optionally utilized.

The compositions of the invention may also be formulated as hydrogels as described in U.S. Pat. No. 6,723,304, describing the production of diglycerol-based hydrogels for oral mucosal use. Gel forming agents useful for preparation of the formulation described herein include carboxmethyl cellulose, sodium hydroxymethyl cellulose, hydroxyethyl cellulose, hydroxypropyl guar, hydroxyethyl starch, polyvinyl pyrrolidone, tragacanth, agar, carrageenan, gum arabic, xanthan gum, guar gum, locust bean gum, carboxyvinyl polymers, fumed silica, silica clays and combinations thereof.

For severe periodontitis, the compositions of the present invention may also be formulated into polymeric gels for introduction and retention within diseased periodontal suclci. DIM, or a DIM-related indole, in a concentration of 0.5-3.0% by weight of the formulation, can be incorporated in long lasting polymeric gels. Such solidifiying, ploymeric gels may also contain anti-NF-kappa B agents and can be manufactured as described in U.S. Pat. No. 5,368,859, “Biodegradable system for regenerating the periodontium”.

### 4.5.2.4 DIM Formulated for Oral Topical Administration Directly to Oral Mucosa in the Form of Mouthwash/Rinse

Representative methods for production of a mouth wash or mouth rinse are described in U.S. Patent Application Publication No. 2004/0076591 A1 “Cyclodextrins in Dental Products, and U.S. Pat. No. 6,261,540, “Cyclodextrins and hydrogen peroxide in dental products”; U.S. Pat. No. 6,534,042, “Taste masking of phenolics using citrus flavors”; U.S. Pat. No. 5,716,601, “Oral compositions, such as oral gels and toothpastes, containing a novel abrasive.”; and U.S. Pat. No. 5,281,410 “Methods of reducing plaque and gingivitis with reduced staining”. In an alternative method, formulations use particle formation steps as described in U.S. Patent Application Publication No. 20030152629.

Representative steps for manufacture of a DIM-containing mouthwash include the following. A DIM mouthwash is formulated by dispersing microcrystalline DIM, Resveratrol, and Curcumin each at 0.5% wt/vol of final mixture in ethanol. Tea tree oil (weight percent 0.01), eucalyptol (weight percent 0.5), and menthol (weight percent 0.5), sucralose (weight percent 0.5 carboxymethyl cellulose (weight percent 6) are then added to the ethanol. The mixture is mixed using a HOCKMEYER HVI Mixer, (Hockmeyer Equipment Co., Elizabeth City, N.C.). Water (sufficient to dilute ethanol to a final concentration of 15% by volume), sorbitol (5% of final volume), glycerine (5% of final volume), and hydroxypropyl-beta-cyclodextrin—weight percent 4) are added and the mixture mixed for 30 minutes. Optionally, Evodiamine and Honokiol, each present at 0.01-0.5% of the total formulation weight are utilized.

### 4.5.2.5 DIM Formulated for Oral Topical Administration Directly to Oral Mucosa in the Form of Chewing Gum

In order to prepare the present compositions into a chewing gum, DIM, or DIM-related indole, alone or combined with an antiflammatory agent and/or antibacterial agent, may be combined with conventionally used carriers including one or more natural or synthetic elastomers, optionally supplemented with one or more solvents, plasticizers or fillers. Natural elastomers suitable for use include substances of vegetable origin such as chicle, jelutong, gutta percha, guayule and crown gum. Examples of synthetic elastomers include butadiene-styrene copolymers, isobutylene-isoprenecopolymers, polyethylene, polyisobutylene, polyvinylacetateand combinations thereof. The elastomer generally comprises from about 14% to about 50% by weight of the composition. Solvent may additionally be added to soften the elastomer component. Suitable solvents include methyl, glycerol or pentaerythritol esters of rosins or modified rosins, such as hydrogenated, dimerized or polymerized rosins as well as terpene resins such as polyterpene. Specific examples of such solvents include pentaerythritol ester of wood rosin, glycerol ester of partially dimerized or polymerized rosin, glycerol ester of tall oil rosin or wood rosin, and partially hydrogenated methyl ester of rosin. Such solvents may be used in an amount ranging from about 5% to about 25% of the composition. DIM or DIM-related indole may be complexed with cyclodextrins or dissolved in such solvents or micronized and suspended in the solvent or elastomer phase of the preparation. Plasticizers, softeners or emulsifiers may also be included in the gum composition in an amount of up to about 30% by weight of the composition. Examples of these components include lanolin, lecithin, glyceryl monostearate, stearic acid, sodium stearate, postassium stearate, glyceryl triacetate, triacetin and glycerin, as well as natural waxes, petroleum waxes, paraffin waxes and microcrystalline waxes to improve texture and consistency.

Representative methods for formulations of chewing gums are described in U.S. Patent Application Publication No. 20030072841, “Polybutene containing chewing gum and confection”.

### 4.5.3 Use of DIM, DIM-Related Indoles and Selected Phytoestrogens in Fortified Foods.

The compositions of DIM, and DIM-related indoles, of the present invention are also utilitized as added ingredients to certain foods to facilitate convenient and regular consumption to promote bone health. Fortified foods include “medicinal foods” which require use under a doctor's care and “functional foods” available to consumers as unregulated specialized foods. Such uses in fortified or “functional” foods typically apply to Food Bars, Drink Mixes, Vegetable Juices, Pasta Mixes, Dry Cereal, Meal Replacement Powders, and Baked Goods. Such uses require specialized production with the dose of DIM in accordance with principles of Generally Regarded As Safe (GRAS) food ingredients. In such applications DIM is added to food products or mixes alone or in combination with selected phytoestrogen formulations, particularly genistein.

Food Bar Products are produced according to the present invention according to known manufacturing and baking practices. Detailed of food bar composition and manufacturing techniques useful with DIM, DIM-Related Indoles, and DIM combined with selected phytoestrogen such as genistein are specified in US. Patent Application Publication Nos. 20030068419 entitled “Food bar compositions”, and 20020168448 entitled “Nutritional food bar for sustained energy”.

Drink Mix Products are produced according to the present invention according to known manufacturing practices. Detailed drink mix composition and manufacturing techniques useful with DIM, DIM-Related Indoles, and DIM combined with selected phytoestrogen such as genistein are specified in US. Pat. No. 6,599,553 by Kealey, et al., entitled “Dry drink mix and chocolate flavored drink made therefrom”. In preferred embodiments, DIM is incorporated in fortified foods, such as drink mixes and food bars, during food production using a particulate form of DIM that is formulated for enhanced absorption (BioResponse-DIM [BioResponse, LLC, Boulder, Colo.]). Genestein is added as a powdered formulation of pure isoflavone (Bonistein [DSM Nutritional Products]). Typically, the DIM is provided in a dose of 10-25 mg/serving (40-100 mg/serving of BioResponse-DIM). Genistein is provided in a dose of 25-100 mg/serving as Bonistein.

### 4.6 Additional Combination Therapy

A number of anti-inflammatory and anti-bacterial compounds can be used in the methods and compositions of the invention in combination with DIM, or a DIM-related indole, with or without a phytoestogen, for the prevention and/or treatment of an oral mucosal disorder or for the promotion of bone health. Non-limiting examples are provided in the subsections below. It is understood in the art that a compound may have both anti-inflammatory and anti-bacterial activity.

### 4.6.1 Anti-Inflammatory Compounds

DIM has been found to have anti-inflammatory activity. When DIM, or a DIM-related indole, is to be used in combination with an anti-inflammatory compound in the methods and compositions of the invention, it is understood that the anti-inflammatory compound to be used is a compound other than DIM, or a DIM-related indole. Anti-inflammatory compounds, unrelated to DIM, having anti-inflammatory activity relevant to treating oral mucosal disorders and/or aging-related and inflammation-associated bone loss and suitable for use in the methods and compositions of the invention are described below.

Optimal anti-inflammatory compounds are selected based on their ability to inhibit inflammation associated with activation of cellular Nf-kappa b. Preference for use of anti-inflammatory compounds is also based on demonstrated anti-inflammatory activity in test systems specific for Nf-kappa b inhibition known in the art (Katula K S, McCain J A, Radewicz A T. Relative ability of dietary compounds to modulate nuclear factor-kappaB activity as assessed in a cell-based reporter system. J Med Food. 2005 Summer; 8(2):269-74). The preferred anti-inflammatory compounds to be used with DIM are safe for oral use, and most preferably safe for ingestion as indicated by a safe history of use as a component of food, as dietary supplements, as herbal remedies, or as established pharmaceuticals.

Resveratrol (3,4′,5-Trihydroxy-trans-stilbene), a natural stillbene, and its dimeric form, viniferin, and derivatives, such as piceatannol (3,4,3′,5′-tetrahydroxystilbene), oxyresveratrol (2,3′,4,5′-tetrahydroxystilbene), 4,4′-dihydroxystilbene, and the alpha- and beta-glucoside, galactoside and mannoside derivatives, such as piceid, have been described for treatment of periodontitis resulting from tobacco use. See, e.g., U.S. Pat. No. 6,716,883. In experimental conditions using human endothelial cells, Resveratrol has been shown to influence the NF-kappa B pathway at physiologic concentrations (Pellegatta et al., Different short- and long-term effects of resveratrol on nuclear factor-kappaB phosphorylation and nuclear appearance in human endothelial cells. Am J Clin Nutr. 2003; 77(5):1220-8). Resveratrol is commonly isolated from grapevines (Vitis vinifera L) and from Polygonium cuspidatum Sieb. Et Zucc (Japanese knotweed). Dried extracts of Vitis vinifera and Polygonium cuspidatum are useful sources for resveratrol for use in the compositions of the present invention. Extracts contain compounds related to resveratrol such as viniferins, piceatannol (3,4,3′,5′-tetrahydroxystilbene), oxyresveratrol (2,3′,4,5′-tetrahydroxystilbene), 4,4′-dihydroxystilbene, and cis- and trans-piceids which are also preferred anti-inflammatory agents for use in the present invention. The naturally occurring and related, synthetic resveratrol derivatives of possible use in the present invention have been described (Aggarwal et al., Role of resveratrol in prevention and therapy of cancer: preclinical and clinical studies. Anticancer Res. 2004; 24:2783-840).

Further anti-inflammatory compounds for use in the present invention include Silibinin and Curcumin. Silibinin (Silybin [3,5,7,-trihydroxy-2-[3-(4-hydroxy-3-methoxyphenil)-2-hydroxymethyl-1,4-benxodioxan-6-il]-chronan-4-one], a flavolignan isolated from the fruits of Silibum marianum (Milk Thistle). Silibinin inhibits constitutive. NF-kappaB activation in cell culture of endothelial cells resulting in a significant decrease in the nuclear level of p65 subunit of NF-kappa B (Yoo et al., Involvement of NF-kappaB and caspases in silibinin-induced apoptosis of endothelial cells. Int J Mol Med. 2004; 13(1):81-6). A further anti-inflammatory compound useful in the present invention is Curcumin. Curcumin is a compound isolated from tumeric, also known as Curcuma domestica, has been proposed for anti-inflammatory uses unrelated to NF-kappaB. These include proposed use in periodontitis as described in U.S. Patent Application Publication No. 20010051184. Derivatives of Curcumin, a soluble curcuminoid, or soluble curcumin derivatives, such as 6-gingerol and 6-paradol, may also be used (Surh et al., Anti-tumor-promoting activities of selected pungent phenolic substances present in ginger. J Environ Pathol Toxicol Oncol. 1999; 18(2):131-9).

Green tea catechins, particulary Epigallocatecin-3-gallate (EGCG), isolated from Camella sinensis, have been shown to have anti-bacterial activity when used intra-orally and to be anti-inflammatory in cell culture. Uses of green tea extracts have been proposed to promote reduced bacterial growth in periodontitis (Hirasawa et al., Improvement of periodontal status by green tea catechin using a local delivery system: a clinical pilot study. J Periodontal Res. 2002; 37(6):433-8).

Caffeic acid (3,4-dihydroxycinnamic acid) and caffeic acid phenethyl ester (CAPE) are structurally related to Curcumin and have NF-kappa B inhibitory activity useful in the present invention. Caffeic acid is an active ingredient of honeybee propolis and a potent and specific inhibitor of NF-kappa B activation. Caffeic acid is also a prominent component of extracts of the leaves of the Simon sweet potato (Ipomoea batatas (L.) Lam: Simon No. 1) where it is present along with chlorogenic acids. Therefore, both Simon extracts and preparations of honeybee propolis are useful components of oral topical compositions and also useful in oral capsule and tablet formulations for the methods of the present invention.

Parthenolide, a sesquiterpene lactone, is found in members of the family Asteraceae, genus Tanacetum (also referred to as the family Compositae), have been used for medicinal purposes for many centuries. The species Tanacetum parthenium, popularly known as feverfew, has been used in folk medicine for the treatment of inflammatory conditions. Extracts of feverfew rich in Parthenolide and pure isolates of Parthenolide are useful as Nf-kappa b inhibitory anti-inflammatory agents in the methods and compositions of the present invention (Jain et al., Antinociceptive and anti-inflammatory effects of Tanacetum parthenium L. extract in mice and rats. J Ethnopharmacol. 1999 Dec. 15; 68(1-3):251-9). Parthenolide-rich extracts and isolates are used in topical formulations and added to tablets and capsules for systemic use in combination with DIM-related indoles.

Also useful as anti-inflammatory agents in the present invention are hexanolic and aqueous extracts from species of Elder Flower or Elderberry (Sambucus nigra L. and Sambucus ebulus L.). These extracts are useful in oral topical formulations for gingivitis and periodontitis in combination with DIM-related indoles (Ebrahimzadeh et al., Antiinflammatory activity of Sambucus ebulus hexane extracts. Fitoterapia. 2006 February ; 77(2):146-8). Liquid Sambucus extracts formulated in combination with DIM-related indoles are particularly useful in oral prepartions for systemic use to treat and prevent oral mucosal disorders in pediatric populations. Sambucus extracts have a safe history of systemic use as folk medicines.

Preparations of Citrus Flavonoids provide an additional source of anti-inflammatory compounds useful in the compositions and methods of the present invention. Oral anti-inflammatory activity of citrus bioflavinoid mixtures have been demonstrated and revealed the presence of hesperidin, naringin and nobiletin. Nobiletin isolated from Citrus depressa is known to inhibit activation of NF-kappa B signaling and is particularly useful in compositions of the present invention (Murakami et al., Effects of selected food factors with chemopreventive properties on combined lipopolysaccharide- and interferon-gamma-induced IkappaB degradation in RAW264.7 macrophages. Cancer Lett. 2003 May 30; 195(1):17-25). Luteolin and Apigenin are useful, structurally-related Citrus Flavonoids which show similar anti-inflammatory activity to Nobiletin. Alcolholic Citrus extracts are particularly useful in oral topical formulations of the present invention since the extracts include penetration enhancing limonene and perrilyl alchol in addition to anti-inflammatory flavonoids. Desirable citrus extract have high flavonoid content, help dissolve poorly soluble DIM-rlated indoles, and increase mucosal penetration of anti-inflammatory agents of the present invention.

Deguelin, an anti-inflammatory agent isolated from several plant species including Mundulea sericea and Derris trifoliata Lour. (Leguminosae), is also useful in the compositions and methods of the present invention. Deguelin, related to rotenone but less toxic, is particularly useful in oral topical formulations in combination with DIM-related indoles based on anticipated synergitic anti-inflammatory activity. Deguelin has recently been shown to be a unique inhibitor of Nf-kappa B mediated inflammation (Chen et al., Deguelin inhibits expression of IkappaBalpha protein in Raji and U937 cells. Acta Pharmacol Sin. 2006 April; 27(4):485-90).

Guggulsterone (4,17(20)-pregnadiene-3,16-dione), a plant sterol derived from the gum resin (guggulu) of the tree Commiphora mukul, is a phytochemical with anti-inflammatory activity also useful as an oral topical and systemic anti-inflammatory agent in the present invention. The resin has been used in Ayurvedic medicine for centuries to treat a variety of ailments, including bone fractures, arthritis, inflammation, and cardiovascular disease. Extracts and/or the gum resin can be used in toothpaste, oral topical gel, chewing gum, and in oral supplements for systemic use. Guggulsterone is known to inhibit activation of NF-kappa B (Shishodia S, et al., Guggulsterone inhibits NF-kappaB and IkappaBalpha kinase activation, suppresses expression of anti-apoptotic gene products, and enhances apoptosis. J Biol Chem. 2004 Nov. 5; 279(45):47148-58).

Sanguinarine is an alkaloid isolated from medicinal plants Sanguinaria canadensis and Chelidonium majus (Papaveraceae) which has proven useful in topical uses for gingivitis. Sanguinarine and related chelerythrine are quaternary benzo[c]phenanthridines which exhibit antimicrobial and anti-inflammatory activities. However, chronic use of oral care products containing 0.03% of sanguinaria has been associated with increased prevalence of maxillary vestibule leukoplakia, a precancerous condition (Mascarenhas A et al., 2002, The association between Viadent use and oral leukoplakia—results of a matched case-control study. J Public Health Dent. 62:158-62). The use of low doses of this compound in the methods and compositions of the invention is preferred.

Useful anti-inflammatory compounds for use in the methods and compositions of the invention include, but are not limited to, pharmaceutically acceptable forms of Evodiamine, an indole alkaloid component extracted from the fruit of Evodiae Fuctus (Evodia rutaecarpa Benth.), ursolic acid, and boswellic acid derivatives or extracts of Frankensence containing boswellic acid. Boswellic acid derivatives are described in U.S. Patent Application Publication No. 20020010168. Also useful are Aloe extracts (Aloe barbadensis, Aloe capensis, and Aloe vera), Allyl Disulfide, Andrographolide, Dehydro-Andrographolide, Deoxy-Andrographolide, Brassinin, Caffeic acid, Capsanthin, Capsaincin, L-Carnitine, L-Carnitine HCl, Carnosic acid 90%, Chelerythrine Chloride, Cromolyn sodium, Deguelin, Diallyl disulfide, Diallyl sulfide 97%, Diallyl trisulfide, Dibenzoylmethane, Ebulin 1, Ellagic acid, (−)Epicatechin, (−)Epicatechin gallate, (−) Epigallocatechin, Epigallocatechin gallate, Ferulic acid, Genistein, 18β-Glycyrrhetinic Acid, Glycyrrhizic acid ammonium salt trihydrate, Green tea polyphenols, Guggulsterone, Honokiol, 5-Hydroxy-L-tryptophan, Hypericin, Hypocrellin A, Ibuprofen, Idebenone, Luteolin, D-Limonene, Limonin, Limonin Glucoside, DL-α-Lipoic acid, Melatonin, Nobiletin, Parthenolide, Perillyl Alcohol, Phenylbutyrate, Phenylethyl 3-methylcaffeate, Phenylethyl 4-methylcaffeate, Phenyl isothiocyanate, Phytic Acid 40-50 wt % aqueous solution, Resveratrol, 9-cis-Retinoic acid, 13-cis-Retinoic acid, trans-Retinoic acid, all-trans-Retinol, retinyl acetate, Retinyl palmitate, Rosmarinic acid, Rutaecarpine, sulforaphane, L-Theanine, Trichostatin A, ursolic acid, Vitamin K3, and Xylitol.

Some anti-inflammatory drugs are useful as additional anti-inflammatory agents in the methods and compositions of the invention. Suitable anti-inflammatory drugs include ibuprofen, flurbiprofen, ketoprofen, aspirin (Acetylsalicylic Acid), salycylamide, kertorolac, naproxen, indomethacin, piroxicam, and meclofenamic acid. Further useful nutritional compounds inhibitory to NF-kappa B include N-Acetyl-L-Cysteine, selenium, selenomethionine, zinc salts, particularly Zinc citrate and Zinc gluconate, and Vitamin-C (L-ascorbic acid), and Vitamin-C derivatives and esters, including L(+)-Ascorbic Acid and Ascorbyl palmitate.

In a preferred embodiment, the anti-inflammatory agent is resveratrol, an extract of Polygonium cuspidatum, silibinin, an extract of Silybum marianum, curcumin, an extract of Curcuma domestica, apigenin, aloe extract, terpenes, particularly the sequiterpene lactone, Parthenolide, citrus extracts, particularly the citrus flavonoid, nobiletin, boswellic acid, caffeic acid, and propolis extracts containing caffeic acid phenthylester, deguelin, extracted from various plant sources including Munduelea sericea, a green tea polyphenol, evodiamine, guggulsterone, sanguinarine, an Andrographis extract, a black tea extract, pomegranate fruit extract, ursolic acid, zinc derivative, or ascorbic acid derivative.

### 4.6.2 Anti-Bacterial Compounds

Compounds which provide anti-bacterial activity to be used in the methods and compositions of the invention include, but are not limited to, tea tree oil, neem oil, manuka oil, eucalyptus oil, lavandula oil, rosmarinus oil, green tea extracts (Camilla sinensis [Epigallocatecin-3-gallate]), perilla seed extracts (Perilla Fructescens Japanica), grapefruit seed extract (cirus Grandis), Magnolia Grandiflora Seed Extract (Honokiol and Magnolol), Stevia extract, extract of Prunella vulgaris L. (Labiatae), Isoquinoline alkaloids from Macleya cordata R. Br. (Papaveraceae) and chitosan. A major constituent of P. vulgaris is rosmarinic acid, a phenolic antioxidant. Other useful antibacterial and anti-plaque agents include triclosan, apigenin, stevia, sanguinarine and sanguinaria, quaternary ammonium compounds, cetylpyridinium chloride, tetradecylpyridinium chloride and N-tetradecyl-4-ethylpyridinium chloride, benzalkonium chloride, bisquanides, chlorhexidine, chlorhexidine digluconate, hexetidine, octenidine, alexidine, halogenated bisphenolic compounds, 2,2′-methylenebis-(4-chloro-6-bromophenol), 5-chloro-2-(2,4-dichloropheno-xy)-phenol, salicylanilide, domiphen bromide, delmopinol, octapinol, other piperadino derivatives, nicin, zinc stannous ion agents, analogs and salts of the foregoing, and mixtures of the foregoing.

Citrus oils are rich in limonene and perrilyl alcohols which can help dissolve other poorly soluble agents including flavones, alkaloids, DIM, and DIM-related indoles. Citrus extracts, and the fraction of honey comb known as propolis, are also a source of terpenes which may contribute to anti-microbial activity. Terpenes have been described for use in oral health products as in U.S. Patent Application Publication Nos. 20040057908 and 20040258633.

### 4.6.3 Medications for Age-Related Bone Loss

In certain embodiment, DIM, or a DIM-related indole, alone or in combination with a phytoestrogen can be administered with medications for age-related bone loss, including both antiresorptive and anabolic types. Antiresorptive medications—estrogens, selective estrogen receptor modulators (raloxifene), steroid derivatives (tibolone), bisphosphonates (alendronate, risedronate, and ibandronate) and calcitonins—work by reducing rates of bone remodeling. Teriparatide (parathyroid hormone) is the only anabolic agent currently approved for osteoporosis in the United States (Kirk D, Fish S A. Medical management of osteoporosis. Am J Manag Care. 2004 July; 10(7 Pt 1):445-55).

### 4.7 Additives

Compositions of the present invention may further include solubility enhancers and mucosal penetration enhancers. This includes the addition of pharmaceutically acceptable cyclodextrins both for DIM formulation and to improve solubility of other anti-inflammatory and anti-bacterial agents, use of methycellulose derivatives, essential oils, including limonene, perrillyl alcohol and terpene derivatives which help to dissolve DIM-related indoles and improve mucosal penetration. Particularly useful are chitosan derivatives which possess both bioadhesive and mucosal penetration-enhancing activity (Sandri et al., Assessment of chitosan derivatives as buccal and vaginal penetration enhancers. Eur J Pharm Sci. 2004; 21(2-3):351-9, and Lim et al., In vivo and in vitro characterization of novel microparticulates based on hyaluronan and chitosan hydroglutamate. AAPS PharmSciTech. 2001; 2(4):20). Also included as mucosal penetration enhancers are SEPA derivatives which are condensates of ethylene glycol and decyl aldehyde (decanal), particularly Sepa 0009 (2-n-nonyl-1,3-dioxolane) and other members of the group of alkyl-substituted acetals and cycloacetals (1,3-dioxolanes).

Surfactants can also be employed in the various oral topical compositions. Any of a variety of types of surfactants can be utilized, including anionic, nonionic, cationic and zwitterionic or amphoteric surfactants, or combinations thereof. Exemplary anionic surfactants include, without limitation, sodium lauroyl sarcosinate, a-olefin sulfonate, taurate, lauryl monoglyceride sulfate, lauryl monoglyceride sulfonate, and combinations thereof. Exemplary nonionic surfactants include, without limitation, TWEEN, lauroyl diethanol amide, stearyl monoglyceride, sucrose fatty acid esters, lactose fatty acid esters, lactitol fatty acid esters, maltitol fatty acid esters, polyoxyethylene sorbitan monostearate, Vitamin E polyethylene glycol esters (Vitamin E tocopheryl succinate polyethylene glycol 1000 (TPGS) [Eastman]), and combinations thereof. Exemplary ampholytic surfactants include, without limitation, betain and amino acid type surfactants. Surfactants can be present in amount of about 0.5 to about 15 weight percent, more typically about 0.5 to about 10 weight percent. Sodium Laureth Sulfate is specifically excluded due to its mucosal damaging activity.

In addition, the oral compositions can include a number of additives, including without limitation, an abrasive agent, a gelling agent, a humectant, a cariostatic agent, a flavoring agent or sweetener, a desensitizing agent, an anti-calculus agent, a whitening agent, a binding agent, a preservative, an opacifying agent, a coloring agent, a buffering agent, or combinations thereof.

Abrasive agents are typically employed in dentifrice compositions. Suitable abrasive agents include, without limitation, aluminum oxide, aluminum hydroxide, calcium hydrogen phosphate dihydrate or anhydride, silica gel, zirconosilicate, silicic anhydride, aluminosilicate, calcium carbonate, calcium pyrophosphate, aluminum silicate, insoluble sodium metaphosphate, magnesium tertiary phosphate, magnesium carbonate, calcium sulfate, synthetic resins, and combinations thereof. Preferred abrasives are Sylodent® 700 and Sylox® from W.M. Grace and Co. Abrasives can generally be employed in effective amounts of between about 10 to about 60 weight percent, more typically about 20 to about 40 weight percent for dentifrices.

Gelling agents (i.e., thickeners) can be used in various compositions to assist in processing. Suitable gelling agents include, without limitation, glycerin, polyethylene glycol, Sylodent, Sylox, carrageenan, sodium carboxymethyl cellulose, alkali metal alginates such as sodium alginate, gums, polyvinyl alcohol, and vee gum. Other desirable gelling agents with desirable mucosal bioadhesive qualities include agar, alginic acid, alginate, amylose, high amylose starch, gum arabic, carrageenan, processed euchema seaweed, casein, carboxymethyl cellulose (CMC), carboxyvinyl copolymer, hydroxypropylcellulose (HPC), hydroxypropylmethylcellulose (HPMC), hydroxyethylcellulose (HEC), methyl cellulose, microcrystalline cellulose (MCC), sodium carboxymethyl cellulose, natural celluloses, chitin, chitosan, collagen, dextran, polydextran, elsinan, gelatin, gellan gum, guar gum, gelatin, ghatti gum, karaya gum, gluten, konjac, levan, locust bean gum, maltodextrin, methylmethacrulate copolymer, oat gum, pectin, low methoxy pectin, polyethylene glycol, polylysine, polybrene, polyvinylpyrrolidone, polyvinyl alcohol, polyacrylic acid, propylene glycol, protein, pullulan, starch, modified starches, soy protein, tara gum, tamarind gum, tragacanth, whey protein, xanthan gum, and zein. Typically, the gelling agents are employed in amount of about 0.3 to about 30 weight percent.

Humectants can also be employed in the oral compositions, particularly toothpastes and gels and oral rinses. Humectants help keep oral care compositions, such as pastes, from hardening upon exposure to air, give oral care compositions a moist feel to the mouth, and may impart desirable sweetness. Suitable humectants include sorbitol, glycerol, propylene glycol, 1,3-butylene glycol, polyethylene glycol, xylitol, maltitol, lactitol, or the like. The humectant can also be used as the bulk carrier in many instances, in which case it can be present in an amount of about 5 to about 90 weight percent, more typically about 5 to about 60 weight percent.

Cariostatic agents can be provided in each form of the oral composition. Suitable cariostatic agents include, without limitation, sodium fluoride, stannous fluoride, aminefluorides, sodium monofluorophosphate, sodium trimeta-phosphate, triclosan, casein, or combinations thereof. If desired, the cariostatic agent can be present in an amount between about 0.01 to about 3 weight percent, more typically between about 0.02 to about 1 weight percent.

Flavoring agents are desired in most oral compositions to enhance the flavor and palatability of the oral composition and, thus, the likelihood of their use. Suitable flavoring agents can be flavoring oils (e.g., oil of spearmint, peppermint, wintergreen, sassafras, clove, sage, stevia extracts, eucalyptus, cinnamon, lemon, and orange, methyl salicylate, etc.) or sweeteners (e.g., sucrose, sucralose, lactose, maltose, xylitol, sodium cyclamate, perillartine, aspartyl phenyl alanine methyl ester, saccharine, etc.). Flavoring agents can be present, either individually or collectively, in an amount of about 0.1 to about 10 weight percent, more typically about 0.1 to about 5 weight percent. Flavoring agents can be complexed with cylodextrins to improve solubility and stability.

Desensitizing agents can be introduced in some embodiments of the oral composition to treat individuals whose teeth are sensitive to thermal shock, chemicals, etc. Suitable desensitizing agents include, without limitation, potassium citrate, potassium chloride, potassium tartrate, potassium bicarbonate, potassium oxalate, potassium nitrate, and strontium salts. Desensitizing agents can be present, either individually or collectively, in an amount of about 0.1 to about 5 weight percent, more typically about 0.1 to about 3 weight percent.

Additional anti-calculus agents can be introduced in some embodiments of the oral composition to treat tartar formation. Suitable anti-calculus agents include, without limitation, alkali-metal pyrophosphates, hypophosphite-containing polymers, organic phosphonates, phosphocitrates, and combinations thereof. Anti-calculus agents can be present, either individually or collectively, in an amount of about 0.1 to about 5 weight percent, more typically about 0.1 to about 3 weight percent.

Whitening agents can be employed in some forms of the oral composition. Suitable whitening agent including Perlite, urea peroxide, calcium peroxide, carbamide peroxide, and hydrogen peroxide. Whitening agents can be employed in amounts of about 0.5 to about 10 weight percent.

Preservatives can be utilized to enhance the storage properties of the topical oral composition. One suitable preservative is benzoate (e.g., sodium benzoate), which also possesses a degree of cariostatic activity.

Opacifying agents can also be added to various oral compositions of the present invention, particularly oral gels and pastes. Titanium dioxide is a white powder which adds opacity to the compositions. Titanium dioxide can be present in an amount of about 0.25 to about 5 weight percent.

Coloring agents may also be added to the oral compositions of the present invention. The coloring agent may be in the form of an aqueous solution, i.e., an approximately 1 percent coloring agent in water solution. Color solutions can be present in an amount of about 0.01 to about 5 weight percent.

Topical oral compositions may also include buffers and salts to buffer the pH anionic strength of the oral composition, thereby promoting its stability. The pH of such oral compositions of the invention is generally in the range of about 4.5 to about 9 or 10, preferably about 6.5 to about 7.5 or 8. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc.).

The alcohol in the various compositions of the present invention must be nontoxic. Preferably the alcohol is ethanol. Ethanol is a solvent and also acts as an antibacterial agent and as an astringent but must be kept to concentrations below 25%.

Suitable viscosity modifiers can be added to the compositions of the present invention. These viscosity modifiers include, polybutene, mineral oil, oregano modified clays, petrolatum, silicas, and mixtures thereof. In one embodiment the viscosity modifier is silica. Where incorporated, the viscosity modifier is present in the polybutene component of the present invention at a level of from about 0.001% to about 30%, in one embodiment from about 0.01% to about 10%, and in another embodiment from about 0.1% to about 3% of the second layer composition.

For use in treating oral mucosal disorders, the present invention provides in another of its aspects an article of manufacture which includes packaging material contained within which is a pharmaceutically acceptable composition of DIM, or DIM-related indole, that is effective to treat oral mucosal disorders. The packaging material comprises a label which indicates that the composition can be used to treat an oral mucosal disorder.

### 4.7.1 Carriers and Excipients

Nutritional and pharmaceutical compositions according to the present invention preferably comprise one or more nutritionally or pharmaceutically acceptable carriers and the active constituents, e.g., a DIM-related indole and, optionally, one or more of a selected phytoestrogen preparation, an additional anti-inflammatory agent or an antibacterial agent. The carrier(s) must be “acceptable” in the sense of being compatible with the other ingredients of the composition and not deleterious to the recipient thereof. In this context, the term “pharmaceutically acceptable” means acceptable for use in the pharmaceutical and veterinary arts, i.e., a carrier which is non-toxic and which does not adversely affect the activity of Diindolylmethane (DIM) and DIM-related indole to treat oral mucosal disorders and/or promote bone health. The term, “nutritionally acceptable” means acceptable for use in a food or drink.

The term “carrier” refers to a diluent, adjuvant, excipient, or vehicle with which the therapeutic is administered. The carriers in the nutritional or pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin; and/or a flavoring agent, such as peppermint, methyl salicylate, or orange flavoring. Calcium or magnesium salts are preferred carriers for the present invention since they may support therapeutic activity of DIM-related indoles.

Nutritional and pharmaceutically acceptable carriers useful to prepare the present compositions for oral and topical administration include conventional carriers used in formulating alcohol-soluble drugs, gel forming polymers including methyl cellulose derivatives, and gel compatible bioadhesives, including chitosan dervivatives.

Preparation of the oral compositions of the present invention can be carried out according to known techniques and procedures, depending upon the particular type of vehicle employed. Where solubility is of concern, suitable cyclodextrins and surfactants can be employed to enhance the solubility of the active ingredients in the selected carrier. However, due to the poor solubility of DIM, DIM-related indoles, and many of anti-inflammatory agents, cyclodextrins are preferred compounds to increase solubility. Cyclodextrins are known to form inclusion complexes with various compounds. The cyclodextrin molecule consists of glucopyranose units arranged in a torus-like or donut-like configuration having all the secondary hydroxyl groups located on one side of the torus and all primary hydroxyl groups located on the other side. Alpha, beta, and gamma cyclodextrin contain 6, 7 & 8 cyclic glucopyranose units, respectively, in the torus shell. The “lining” of the internal cavity is formed by hydrogen and glucosidic oxygen-bridge atoms and therefore the surface is slightly apolar.

Therapeutic formulations suitable for oral administration, e.g., tablets and pills, may be obtained by compression or molding, optionally with one or more accessory ingredients. Compressed tablets may be prepared by mixing phytochemicals, and compressing this mixture in a suitable apparatus into tablets having a suitable size. Prior to the mixing, the DIM-related indole may be mixed with a binder, a lubricant, an inert diluent and/or a disintegrating agent.

In a preferred embodiment, the DIM-related indole is mixed with a binder, such as microcrystalline cellulose, and a surfactant, such as sodium lauryl sulphate, until a homogeneous mixture is obtained. Subsequently, another binder, such as polyvinylpyrrolidone (polyvidone), is transferred to the mixture under stirring with a small amount of added water. This mixture is passed through granulating sieves and dried by desiccation before compression into tablets in a standard tableting apparatus.

A tablet may be coated or uncoated. An uncoated tablet may be scored. A coated tablet may be coated with sugar, shellac, film or other enteric coating agents.

When the pharmaceutical composition is a capsule, it may contain a liquid carrier, such as a fatty oil, e.g., cacao butter.

Suitable nutritional and pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. The composition can be formulated as a suppository, with traditional binders and carriers such as triglycerides.

Compositions of the present invention may further include solubility enhancers and skin penetration enhancers. This includes the addition of pharmaceutically acceptable cyclodextrins both for DIM oral formulation and to improve solubility of added phytoestrogens, use of methycellulose derivatives, essential oils, including limonene, perrillyl alcohol and terpene derivatives. Flavoring agents can be complexed with cylodextrins to improve solubility and stability for fortified food applications.

In yet another embodiment, the therapeutic compound can be delivered in a controlled release system. In one embodiment, a pump may be used (see Langer, supra; Sefton, CRC Crit. Ref. Biomed. Eng. 1987, 14:201; Buchwald et al., Surgery 1980, 88:507; Saudek et al., N. Engl. J. Med. 1989, 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 1983, 23:61; see also Levy et al., Science 1985, 228:190; During et al., Ann. Neurol. 1989, 25:351; Howard et al., J. Neurosurg. 1989, 71:105).

Other controlled release systems are discussed in the review by Langer (1990, Science 249:1527-1533).

A number of references have been cited, the entire disclosures of which are incorporated herein by reference.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

### 5 EXAMPLES

### 5.1 Example 1

### Manufacture of Processed DIM for Enhanced Oral Bioavailability

Preparation of processed Diindolylmethane is accomplished according to the steps outlined in U.S. Pat. No. 6,086,915, herein incorporated by reference in its entirety. Briefly, this included mixture of about 10-40% by final weight of Diindolylmethane with about 10-40% by final weight of vitamin E polyethylene glycol 1000 succinate (Vitamin-E-TPGS, Eastman Chemical), 2-20% by final weight phosphatidyl choline (Phospholipon 50G, Rhone Poulenc) and 15-30% by final weight hexanol. This mixture is made homogeneous by mixing. The homogeneous mixture of indoles and other oil soluble substituents listed above is added to a solution of modified starch in water (Capsul Starch from National Starch, Inc.). The starch component forms from 30-70% of the final dry weight of the product. The well dispersed final combined mixture is then subjected to spray drying. The resultant product is a fine powder containing Diindolylmethane contained within the starch particles.

### 5.2 Example 2

### Manufacture of Capsules Containing Diindolylmethane

Capsules containing 150-300 mg of processed Diindolylmethane, as produced according to the steps described in Example 1, are made by mixing the processed Diindolylmethane with microcrystaline cellulose and placing the mixed powder into opaque gelatin capsules.

Capsules containing the combination of 150 mg of processed Diindolylmethane and 30 mg of Resveratrol (from 300 mg of Regrape X) (Interpharma Praha, Czech Republic), are made by mixing the processed Diindolylmethane, Regrape X, with microcrystaline cellulose or rice flour excipient and placing the mixed powder into opaque gelatin capsules.

Capsules containing the combination of 150 mg of processed Diindolylmethane and Silibinin, are made by mixing the processed Diindolylmethane with 200 mg of SiliPhos (Idena, Inc.), adding microcrystaline cellulose or rice flour excipient and placing the mixed powder into opaque gelatin capsules.

Capsules containing the combination of 100 mg of processed Diindolylmethane and 100 mg of genistein (from Bonistein) (DSM Nutritional Products, Holland), are made by mixing the processed Diindolylmethane, Bonistein, with microcrystaline cellulose or rice flour excipient and placing the mixed powder into opaque gelatin capsules.

Capsules containing the combination of 100 mg of processed Diindolylmethane and Ipriflavone, are made by mixing the processed Diindolylmethane with 200 mg of Ipriflavone (3-phenyl-7-propan-2-yloxy-chromen-4-one), adding microcrystaline cellulose or rice flour excipient and placing the mixed powder into opaque gelatin capsules.

Capsules containing the combination of 100 mg of processed Diindolylmethane and equol, are made by mixing the processed Diindolylmethane with 100 mg of equol ((3S)-3-(4-hydroxyphenyl)chroman-7-ol), adding microcrystaline cellulose or rice flour excipient and placing the mixed powder into opaque gelatin capsules.

### 5.3 Example 3

### Manufacture of Complex Particle Formulations with DIM-Related Indoles for Improved Bio-Delivery to Oral Mucosa

Introduction: Topical treatments of oral mucosal disorders according to the present invention are benefited by exposing inflamed oral mucosa to higher concentrations of DIM-related indoles for more prolonged periods of time. Therefore, formulation of DIM, with or without additional anti-inflammatory agents and/or antibacterial agents, in slowly dissolving particles improves the treatment effect. Additional, desirable characteristics for sustained release particles of active agents include bioadhesive and penetration enhancing activity that prolongs contact of the particle with the oral mucosa and improves penetration of active agents into the mucosa. Manufacturing of DIM-containing particles was accomplished according to the following formulation methods to achieve these objectives. This processing includes the use of complex particle formation using methycellulose derivatives (Hydroxypropylmethylcellulose [HPMC]), cyclodextrins (Hydroxypropyl β-cyclodextrin [HPCD]), soluble glucans (Beta-1,3-glucan [BetaPrecise-909 and BetaPrecise-929, Cypress Systems, Inc., Fresno, Calif.]), and chitosan derivatives. Spray dried complex particles were made followed by dissolution testing which demonstrated slower, more prolonged release of dissolved DIM from the particles manufactured with HPMC/HPCD combinations than that seen with particles manufactured with Vitamin E TPGS and simple, unformulated DIM crystals. The HPMC/HPCD DIM particles are preferred for use in oral gels, toothpastes, toothpowders, mouthwashes, and chewing gums of the present invention.

Procedure Used for Forming Particles of DIM, Beta-Cyclodextrin and Hydroxypropyl Methylcellulose: 65 g of dichloromethane (Superior Solvents, lot #AN5213409) was poured into a 250 ml glass beaker. 10.0 g of ethanol (200 Proof, Aldrich lot #01750KC) was added. Mixing using a magnetic spin bar was begun. 1.0 g of HPMC (Pharmacoat 606 [HPMC, Shin-Etsu lot #306305]) was added to the stirring solvent until HPMC dissolved. 1.0 g of DIM Micronized (BioResponse lot #02179) was added to the solution, stirring until it dissolved. 30.0 g of ethanol was poured into a second 100 ml beaker; stirring using a magnetic spin bar was begun. 4.0 g of HPBC (Cavasol W7 HP Pharma [Beta Cyclodextrin, Wacker Fine Chemicals batch #73B009]) was added to the 30 g of ethanol and stirred until HPBC dissolved. Additional ethanol (50 cc) and aliquots of deionized water totaling 5 ml were added to fully dissolve HPBC. The Cavasol solution was transferred into a 400 ml glass beaker. The DIM/HPMC solution was added to the HPBC solution, mixed well and fed to the nozzle system of a mini-spray dryer. The solution was pumped to the nozzle system of the spray drier by a peristaltic pump at a feed rate of 40 ml/minute. The nozzle atomizing air pressure was set at 60 psi. A two-fluid nozzle system with a 35100 nozzle and 120 air cap was employed. There was no heat added to the air of the spray drier. The inlet air temperature at the nozzle system was 34° C. The outlet air temperature was 19° C. The dry, solid particles were collected. Microscopic examination showed smooth, regular particles of 20 micron average diameter.

Subsequently, additional formulations were prepared substituting up to 4.0 g of Chitosan or up to 4.0 g of Beta-1,3-glucan for all or part of the HPMC in the above formulation and particle formation process.

Testing of Dissolution of DIM Containing Particles: In order to determine the release rate profiles of DIM from matrix, particle formulations, a test using pH 6.8 phosphate buffer system containing 0.5% Tween 80 was utilized. The procedure included dissolving an amount of spray dried particles containing a known amount of DIM in a standard dissolution apparatus with standard simulated saliva solution. For the following testing, 900 ml of stock pH 6.8 buffer solution was placed into a round bottom beaker in the dissolution test apparatus. To the solution, 5 g of Tween 80 (Sorbitan Monooleate) was added and mixed while bringing to a temperature of 37° C. To this, 600 mg of a test sample with known DIM content was added to the solution and stirred at 50 rpm. At timed intervals 3.0 ml aliquots of solution was removed and filtered through 0.22 um filter. The absorption of the sample was read at 283 nm on an IR spectrophotometer, blanking vs. pH 6.8 buffer containing 0.5% Tween 80. The percent released DIM compared to the 100% complete, theoretical level was calculated.

The following results were obtained for various complex particle products containing DIM:

TABLE 2

| | Percent DIM Released from Particle | | | |
|---|---|---|---|---|
| | | Micro- | | |
| Time | Vitamin E | crystalline | HPMC | HPMC/HPCD |
| (Minutes) | TPGS¹ | DIM | (Apr. 28, 2003-A) | (Feb. 23, 2005-A) |
| 0 | 0 | 0 | 0 | 0 |
| 15 | 48.2 | 55.1 | 38.9 | 10.1 |
| 30 | 72.6 | 73.6 | 56.6 | 18.4 |
| 45 | 88.6 | 78.8 | 67.5 | 28.5 |
| 60 | 99.1 | 83.5 | 74.5 | 48.1 |

¹Prepared according to U.S. Pat. No. 6,086,915

Further dissolution studies are performed in various dissolution media designed to better resemble human saliva. The conclusion from the above studies is that particle formation with biocompatible, polymeric materials can increase the dissolution time and exposure time of oral mucosa to DIM-related indoles when administered in topical oral formulations.

Conclusions: Particles produced as described containing HPMC, HPBC, Chitosan, and Beta-1,3-glucans are used, according to their content of DIM and other NF-kappa B inhibitors as ingredients in the manufacture of formulations according to the present invention. These particulate complexes are used to prolong and increase exposure of oral mucosa to topically applied DIM and other NF-kappa B inhibitory agents.

### 5.4 Example 4

### Manufacture of DIM Containing Mouthwash

A DIM/alcohol/glycerine suspension is formulated for use as a mouthwash according to the following method:

Disperse microcrystalline DIM, Resveratrol, and Curcumin each at 0.5% wt/vol of final mixture in ethanol. Tea tree oil (weight percent 0.01), eucalyptol (weight percent 0.2), and menthol (weight percent 0.2), suralose (weight percent 0.5 carboxymethyl cellulose (weight percent 6) are then added to the ethanol. Evodiamine and Honokiol, each present at 0.01 of the total formulation weight are then added to the ethanol. The ethanol mixture is mixed using a HOCKMEYER HVI mixer (Hockmeyer Equipment Co., Elizabeth City, N.C.). Water (sufficient to dilute ethanol to a final concentration of 15% by volume), sorbitol (5% of final volume), glycerine (5% of final volume), and hydroxypropyl-beta-cyclodextrin-weight percent 4) are added and the mixture mixed for 30 minutes. A translucent mixture results and is transferred to labeled bottles.

### 5.5 Example 5

### Manufacture of DIM Containing Oral Topical Gel

A DIM-containing oral gel dentifrice is formulated by dispersing carboxymethyl cellulose (weight percent 2) in glycerin (weight percent 15) using a HOCKMEYER HVI mixer, (Hockmeyer Equipment Co., Elizabeth City, N.C.). Water (weight percent 17.5), and sorbitol (weight percent 28) are added and are mixed for 25 minutes. 1% DIM weight percent of final gel (using microcrystalline DIM complexed with hydroxypropyl-beta-cyclodextrin and hydroxypropylmethylcellulose—weight percent of DIM-2) and additional hydroxypropyl-beta-cyclodextrin (weight percent 5) are then added and are mixed for a further 10 minutes. The phenolics are mixed together, i.e., tea tree oil (weight percent 0.5), eucalyptol (weight percent 0.5), and menthol (weight percent 0.5), to make a phenolic phase. The phenolic phase is added to the cellulose/sorbitol/cyclodextrin/water phase until the phenolics are dissolved. Sylodent. 700 (weight percent 14), Sylox (weight percent 6), Green Tea Extract (weight percent 1) are then added and mixed thoroughly for 30 minutes. The resulting light green gel is deaerated to remove air bubbles.

### 5.6 Example 6

### Manufacture of Food Bars Containing Powdered Mixtures of DIM-related Indoles and Genistein

Food Bar Products are produced according to the present invention according to known manufacturing and baking practices. Details of food bar composition and manufacturing techniques useful with DIM, DIM-Related Indoles, and DIM combined with selected phytoestrogen such as genistein are specified in U.S. Patent Applications Publication Nos. 20030068419 entitled “Food bar compositions”, and 20020168448 entitled “Nutritional food bar for sustained energy”.

The following is an illustrative example of production techniques used to produce a nutraceutical food bar to support bone health:

| | |
|---|---|
| Percent by Weight | Formula Range |

| | | |
|---|---|---|
| Food Bar Ingredients | | |
| Binder | 32 | 15-40 |
| Corn Syrup | 14 | 10-20 |
| High Fructose Corn Syrup | 8 | 3-13 |
| Honey | 3 | 1-15 |
| Whey | 1.2 | 1-3 |
| Lecithin (Soy) | .1 | .1-3 |
| Dry Ingredients: | | |
| Rice crisp | 22 | 13-30 |
| Rolled oats | 5 | 2-10 |
| Corn Bran | 2 | 1-8 |
| Soy Nuts | 4 | 2-8 |
| Whey Protein Isolate | 6 | 4-10 |
| Fructose | 2.5 | 2.5-8 |
| Vitamin Mix | 0.16 | 0.1-4 |
| Active Ingredients: | | |
| DIM (BioResponse-DIM) | 75 mg/bar | 50-150 mg/bar |
| Genistein (Bonistein) | 50 mg/bar | 25-100 mg/bar |

The Binder, Corn Syrup, High Fructose Corn Syrup, Honey, Whey and Lecithin are mixed in a kettle and heated to 625° C. The Dry Ingredients and Active Ingredients are added to a ribbon type blender and mixed for 1 minute. The binder is added to the dry mix and again mixed for 1 minute. The mix is fed to a bar forming line. Finished bars are cooled before packaging. Other formulas can be prepared within the indicated ingredient ranges with variations on the presentation of the final product and additions such as fruit flavoring.

### 5.7 Example 7

### Manufacture of Dry Drink Mixes Containing Powdered Mixtures of DIM-Related Indoles and Genistein

Drink Mix Products are produced according to the present invention according to known manufacturing practices. Detailed drink mix composition and manufacturing techniques useful with DIM, DIM-Related Indoles, and DIM combined with selected phytoestrogen such as genistein are specified in U.S. Pat. No. 6,599,553 by Kealey, et al., entitled “Dry drink mix and chocolate flavored drink made therefrom”.

The following is an illustrative example of production techniques used to produce a nutraceutical drink mix to support bone health:

Dry Drink Mix with Cocoa Powder Containing DIM and Genistein.

A dry drink mix containing the cocoa powder of having enhanced levels of cocoa polyphenols was made according to the following formulation:

| | % |
|---|---|
| Inactive Ingredients: | |
| Sucrose | 65.0667 |
| Malt Powder | 11.9122 |
| Cocoa Polyphenol Rich Cocoa Powder | 18.0185 |
| Alkalized Cocoa Powder | 4.0041 |
| Vanillin | 0.0025 |
| Lecithin | 0.9960 |
| | 100.00 |
| Active Ingredients | |
| DIM (BioResponse-DIM) | 100 mg/serving |
| Genistein (Bonistein) | 90 mg/serving |

The dry ingredients are batched according to the above formulation and mixed for one hour in a Kitchen Aid Professional Mixer (Model KSM50P) using a wire whip at #2 speed. The lecithin is agglomerated prior to use in the recipe in a Niro-Aeromatic Agglometer (Model STREA/1). The Active ingredients are added to provide the mg amounts per 2 ounce final portion of the dry ingredients.

The drink mix is added to 8 ounces of water or milk, blended in a kitchen blender, and consumed once daily to promote bone health.

### 5.8 Example 8

### Treatment of Gingivitis in a Subject with Rheumatoid Arthritis

A 49 year old woman with a diagnosis of rheumatoid arthritis was referred for Diinolylmethane (DIM) treatment due to intolerance of oral Plaquenil®. She had experienced unacceptable dizziness taking the Plaquenil®. She had diffuse small joint pain and elevated Rheumatoid Factor and Antinuclear Antigen serum titers. She had a prior history of gingivitis with increased gingival probe depth, bleeding on probing and tenderness of gum tissue. Her baseline periodontal exam showed patches of reddened, tender gingiva with mild swelling. On probing, a total of 12 bleeding sites were documented. She began taking DIM (BioResponse-DIM 150, BioResponse, Boulder, Colo.) taking 50 mg of DIM twice daily. Within one month she noticed diminished join pain. Following three months of treatment, resolution of gingivitis was documented with diminished bleeding and tenderness of her gum tissue documented during re-evaluation by her dentist. The re-exam showed reduced gingival redness, less tenderness, less swelling, and a reduction to 8 bleeding sites on probing. She increased her dose of DIM to 100 mg twice daily and experience further resolution of her joint pain over the next 2 months.

### 5.9 Example 9

### Treatment of Mild Oral Mucosal Inflammation with Topical DIM

An 20 year old male is referred by his dentist for adjunctive treatment of chronic gingivitis. He has moderate gingival inflammation associated with redness and mild swelling. He reports bleeding following brushing of teeth. He begins an oral care program consisting of twice daily application of an oral topical gel containing 2% DIM (using microcrystalline DIM complexed with cyclodextrin [See Illustative Example formulation “A” in Table 1]). In addition, he uses a mouthwash containing 0.5-1.0% DIM (using microcrystalline DIM complexed with cyclodextrin [See Illustative Example formulation “E” in Table 1]) before retiring at night. After 2 months of oral topical use of DIM his signs of oral mucosal inflammation are expected to be diminished.

### 5.10 Example 10

### Topical Treatment of Mild Oral Mucosal Inflammation with Topical DIM Formulation

A teenage girl is referred by her dentist following the diagnosis of chronic gingivitis. At her last dental examination, she was noted to have mild to moderate inflammation of portions of her gum tissue and slight bleeding on probing of periodontal sulci at 50% of sites. She describes blood visible on her tooth brush following brushing. She begins an oral care program consisting of twice daily brushing with a non-fluoride tooth paste (Tom's of Maine; Kennebunk, Me.) followed by post brushing application of an oral topical gel containing 1-2% DIM (using microcrystalline DIM complexed with cyclodextrin) by silibinin (2% by weight SiliPhos), and resveratrol (2% by weight Regrape X), see Illustative Example formulation “B” in Table 1. Application of the oral gel was followed by flossing of teeth to increase exposure of affected mucosa. Re-examination after 6 weeks is expected to reveal diminished oral mucosal inflammation and a history of diminished bleeding associated with brushing teeth.

### 5.11 Example 11

### Treatment of Moderate Oral Mucosal Inflammation in a Subject using Oral Topical DIM

A 45 year old male with the diagnosis of moderate-severe periodontitis is referred for additional care. He smokes 5-10 cigarettes per day. His initial dental examination reveals red, inflamed gingival with swelling and some hypertrophy. On a repeat visit following root scaling, probing of periodontal sulci reveals bleeding in greater than 60% of probing sites with persistent flow of blood. His averaged papillary bleeding score (PBS) of Loesche (Loe et al., The Gingival Index, the Plaque Index and the Retention Index Systems. J Periodontol. 1967; 38(6):Suppl:610-6) is 2.5. His periodontal probing depth averages 4-5 mm. The subject will begin a daily treatment regimen which includes twice daily ingestion of 2 capsules each containing processed DIM (BioRespone-DIM® 150 mg) and silibinin SiliPhos® 200 mg). In addition, the subject will begin a twice daily application by toothbrush of a gel dentifrice containing DIM in a concentration of 1.0-2.0% by weight of the formulation along with SiliPhos® in a concentration of 2% by weight of the formulation, and Regrape X® in a concentration of 2% by weight of the formulation with tea tree oil in a concentration of 0.25% by weight of the formulation. Teeth will be flossed following brushing. Following brushing and flossing, a toothbrush will be used to apply an oral gel containing DIM in a concentration of 1.0% by weight of the formulation. Re-examination after two months of treatment as described is expected to reveal reduced oral mucosal inflammation, a reduced PBS, and diminished average probing depth.

### 5.12 Example 12

### Pilot Clinical Trial Showing the Effectiveness of Toothpaste Containing DIM in Chronic Gingivitis Subjects

A placebo controlled trial will be conducted to show the benefits of DIM containing dentifrice in subjects with chronic gingivitis using a toothpaste formulated with DIM, silibinin, and resveratrol. Subjects meeting the inclusion criteria for chronic ginigivitis will be randomized to use the DIM tooth paste or a placebo toothpaste without DIM, silibinin, and resveratrol. Post-treatment scores for severity of gingivitis will be compared to those at entry to the study.

A total of approximately 40 adult subjects will be selected for the study. They will manifest gingivitis as defined by bleeding on gentle probing at more than 50% of the sites examined. Informed written consent to participate in the study will obtained from each subject. All subjects will be otherwise healthy and will not have undergone any antibiotic or anti-inflammatory therapy in the previous six months.

The DIM toothpaste contains particles of DIM (0.25-1.5%), processed according to U.S. Pat. No. 6,086,915, silibinin added as SiliPhos (0.25-2.0%), and resveratrol (0.25-2.0%) in a gel formulation with tea tree oil (0.25-0.75%), a silica abrasive system, humectants, flavoring, and surfactants, but no fluoride. The placebo toothpaste will contain no DIM, silibinin, or resveratrol, but will be otherwise identical.

At baseline subjects will be examined and the condition of the periodontium will be evaluated using the method of Caton and Polson for a Papillary Bleeding Index (PBI) (Caton et al., Associations between bleeding and visual signs of interdental gingival inflammation. J Periodontol. 1988; 59(11):722-7). The degree of gingivitis will assessed using a non-invasive modification of the Loe-Silness index for a Gingival Index (GI) (Loe, The Gingival Index, the Plaque Index and the Retention Index Systems. J Periodontol. 1967; 38(6):Suppl:610-6). The probing depth for each tooth on lingual and buccal gingival margins will be recorded.

All subjects will be divided randomly to two equal groups. They will be instructed in detail to follow the guidelines of good oral hygiene. After scaling, they will receive the toothpaste containing DIM or placebo according to a random code by which double blinding will be maintained and be given two toothbrushes. Follow-up visits will be conducted at 2-3 and 5-6 weeks, at which time the oral cavity and periodontium will be re-assessed in detail. The data will be analyzed and expressed as mean +S.D.

Any significant differences between the three visits for each index (PBI, and GI) will be analyzed by a one-way analysis of variance (ANOVA).

### 5.13 Example 13

### Demonstration of Bone Supporting Activity of DIM Alone and in Combination with Genistein in an in vitro Bone Cell Culture Model

Introduction:

The activity of DIM as an inhibitor of osteoclast function and survival will be demonstrated in an established culture system for mouse osteoclast-like multinucleated cells (OCLs). Osteoclasts are primary bone-resorbing cells that play a critical role in bone remodeling. This model will be used to demonstrate inhibition of OCL function using a pit-formation assay on dentine slices. Inhibition of osteoclast function and survival supports bone maintenance and formation of new bone. In addition, the combined, additive effect of DIM plus genistein will be demonstrated using the same methods.

Co-culture System and Enrichment of Osteoclast-Like Cells to be Employed.

Osteoblasts will be obtained from the calvariae of newborn ddY mice and bone marrow cells will be obtained from the tibiae of male mice and be cocultured in aMEM (Life Technologies, Grand Island, N.Y.) containing 10% FBS, 1α,25-dihydroxyvitamin D₃ (10⁻⁸ M) (Wako Pure Chemical, Osaka, Japan) and PGE₂ (10⁻⁶ M) (Sigma) in 100-mm-diameter dishes coated with collagen gels (Nitta Gelatin, Osaka). OCLs will be formed within 6 days in culture and will be removed from the dishes by treating with 0.2% collagenase (Wako). The purity of OCLs in this fraction (crude OCL preparation) will be about 5%. To further purify the OCLs, the crude OCL preparation will be replated on culture dishes. After further culture for 8 h, osteoblasts will be removed with PBS containing 0.001% pronase E (Calbiochem, La Jolla, Calif.) and 0.02% EDTA.

Pit Formation Assay

OCLs preparations (15,000 cells/0.1 ml/well) will be seeded on dentine slices (4-mm diameter) which had been placed in 96-well plates. After incubation for 2 h, dentine slices will be transferred to 48-well plates (one slice/well). DIM at various concentrations will be added to certain wells. Genistein alone at various concentrations will be added to certain wells. In a separate experiment, DIM and Genistein at various concentrations will be added to the same wells. Pit formation by OCls will be determined after culture for 24 h and 48 h. For the pit formation assay, cells will be removed from dentine slices, and the resorbed area will be stained with Mayer's hematoxylin. The numbers of pits on the slices will be counted. To quantitate pit-forming activity of OCLs, the areas of resorption lacunae will also be measured with an image analysis system (LA-525; PIAS Co., Tokyo). The results will be expressed as the percentage of resorbed area with respect to the whole surface area of a dentine slice.

Experimental Conditions to be Tested.

1. DIM alone using 0.5-50 micromolar concentrations.

2. Genistein alone using 0.5-50 micromolar concentrations

3. DIM and Genistein in combination using 0.5-50 micromolar concentrations

Anticipated Results and Significance.

DIM alone is expected to demonstrate inhibition of pit formation due to inhibition of osteoclast function and survival. Genistein is expected to show similar activity but require greater concentration for activity than DIM. The combination of DIM and Genistein is expected to show additive activities, permitting reduction of the concentration of DIM and Genistein for maximal inhibition. These findings will support the in vivo use of DIM alone and in combination with Genistein to maintain and support bone health through inhibition of osteoclast function.

### 5.14 Example 14

### Reduction of Osteoclast Numbers in Rats Treated with Absorption-Enhanced DIM for One Year

A one year study was conducted including evaluation of the impact of chronic feeding of DIM and I3C versus control diet in rats. The study used a dose of 20 mg/kg/day DIM, formulated for increased absorption (BioResponse-DIM, BioResponse, Boulder, Colo. [Example 1]), and 50 mg/kg/day Indole-3-Carbinol (I3C) (Sigma Chemical, St. Louis, Mo.). Bone histomorphometry was performed comparing metaphyseal bone in male and female animals using established techiques (Huffer W E, Ruegg P, Zhu J M, Lepoff R B., Semiautomated methods for cancellous bone histomorphometry using a general-purpose video image analysis system. J Microsc. 1994 January; 173 (Pt 1):53-66). Counts of osteoclast number, averaged for male and female groups are summarized in the following chart:

Osteoclast Cell Number in Metaphyseal Bone
After 1 Year DIM or I3C Treatment in Rats

| | | | | | Standard |
|---|---|---|---|---|---|
| | | | | | Error of |
| Sex | N | DIM | I3C | Control | Mean |
| Male | 3 | 9.3* | 14.3* | 10.7 | 1.2 |
| Female | 3 | 14.3 | 18.3 | 17.9 | 3.5 |

*Statistically significant difference between groups (p = .04)

The results showed a reduction in osteoclast number seen only in the DIM treated animals. The reduced numbers of osteoclasts in DIM treated animals compared with I3C treated and Control animals indicates suppression of osteoclast cell population consistent with inhibibition of bone resorptive activity by DIM. Lower oseteoclast numbers in DIM treated animals compared to I3C treated animals indicates a different response of bone to I3C versus DIM treatment. Based on these results DIM has greater potential to support bone health than non-DIM products known to be generated from I3C in vivo (De Kruif et al., Structure elucidation of acid reaction products of indole-3-carbinol: detection in vivo and enzyme induction in vitro. Chem Biol Interact. 1991; 80(3):303-15).

### 5.15 Example 15

### Promotion of Bone Health in a Subject with Rheumatoid Arthritis using Formulated DIM

A 49 year old woman with a diagnosis of rheumatoid arthritis was referred for Diinolylmethane (DIM) treatment due to intolerance of oral Plaquenil®. She had experienced unacceptable dizziness taking the Plaquenil®. She had diffuse small joint pain and elevated Rheumatoid Factor and Antinuclear Antigen serum titers. She had a prior history of gingivitis with increased gingival probe depth, bleeding on probing and tenderness of gum tissue. Her baseline periodontal exam showed patches of reddened, tender gingiva with mild swelling. On probing, a total of 12 bleeding sites were documented. She began taking DIM (BioResponse-DIM 150, BioResponse, Boulder, Colo.) taking 50 mg of DIM twice daily. Within one month she noticed diminished join pain. Following three months of treatment, resolution of gingivitis was documented with diminished bleeding and tenderness of her gum tissue documented during re-evaluation by her dentist. The re-exam showed reduced gingival redness, less tenderness, less swelling, and a reduction to 8 bleeding sites on probing. She increased her dose of DIM to 100 mg twice daily and experience further resolution of her joint pain over the next 2 months. Subsequently she discontinued use of DIM and noted gradual return of painful swollen joints in her hands over 2 months. Resumption of DIM use at 50 mg/day of DIM resulted in diminished pain and swelling of the affected joints after 1 month. Continued use of DIM was associated with further reduced pain and normal appearance and function of the affected joints.

### 5.16 Example 16

### Demonstration of Improved Bone Health in a Placebo Controlled Clinical Study of DIM and Genistein in Postmenopausal Women with Early Osteoporosis

Introduction:

A placebo controlled trial will be conducted to show the benefits of DIM alone and in combination with Genistein in early postmenopausal women with osteopenia and early osetoporosis. Subjects meeting the inclusion criteria will be randomized to use Placebo capsules, DIM capsules, Genistein Capsules, or a combination of DIM capsules and Genistein capsules for a 1 year period of time. The protocol will investigate the impact of DIM, Genistein, and the combination of these supplements on various indicators of bone health. Laboratory methods and clinical trial design will be adapted from prior clinical trials shown to produce relevant information utilizing phytoestrogens (Morabito et al., Effects of genistein and hormone-replacement therapy on bone loss in early postmenopausal women: a randomized double-blind placebo-controlled study. J Bone Miner Res. 2002 October; 17(10):1904-12).

Subjects:

Ethical Committee approval will be obtained for a single center, double-blind, placebo-controlled, randomized study. Participants will be recruited from an established Osteoporosis Clinic. All participants will give informed consent. Participants will be healthy, ambulatory women 47-60 years of age, without surgically induced menopause, and without a menstrual period in the preceding year.

At the beginning of the study, a complete family history, physical examination, laboratory evaluation (chemistry and hematology panel), and measurements of BMD at the lumbar spine and femoral neck will be performed. All the women will undergo a mammography and a transvaginal ultrasound study for determination of endometrial thickness.

Anticipated exclusion criteria will be clinical or laboratory abnormalities that suggest cardiovascular, hepatic, or renal disorders; coagulopathy, use of oral or transdermal estrogen, progestin, androgen, or other steroids in the preceding year; smoking habit of more than two cigarettes per day; previous treatment with any drug that could affect the skeleton; a family history of estrogen-dependent cancer; BMD at the femoral neck >0.795 g/cm². This BMD value corresponds to a T score=−1 SD.

Diet:

All patients will receive dietary instruction for an isocaloric fat-restricted diet offering 30% energy from fat, <10% of energy from saturated fatty acids, and a cholesterol intake of <300 mg/day. To avoid any interference with the possible effects of the different therapeutic interventions on lipid profile, the intake of soy products, legumes, or other nutritional supplements will be prohibited. Dietary calcium intake will be estimated at baseline and after 2, 4, 6, 8, and 10 months by means of a food-frequency questionnaire. Women with a calcium intake of <500 mg/day will be advised to increase their intake. This diet will be continued throughout the study and compliance will be reinforced by a nutritionist.

Treatments:

After a 4-week stabilization on the standard fat-reduced diet, participants to the study will be randomly assigned to receive DIM (50-75 mg twice daily [BioResponse-DIM Capsules, BioResponse, LLC, Boulder, Colo.]), the phytoestrogen genistein (45-90 mg twice daily [Bonistein, DSM Nutritionals]), or twice daily placebo. DIM, genistein, and placebo capsules will appear exteriorly similar. The subjects will complete a baseline quality of life questionnaire including frequency and severity of menopausal symptoms.

Measurements:

The BMD of the anteroposterior lumbar spine and femoral neck will be measured by DexaScan (DXA) at baseline and after 1 year of treatment. The DXA instrument will be calibrated on a daily basis according to the manufacturer's instructions. BMD data will be expressed as grams per squared centimeter.

After an overnight fast, venous blood samples will be drawn between 8 and 9 a.m. The serum will be separated from the blood corpuscles by centrifugation and kept frozen at −70° C. until analysis for calcium (Ca²⁺), bone-specific ALP (B-ALP), osteocalcin (bone Gla protein [BGP]), intact parathyroid hormone (PTH), 25-hydroxyvitamin D₃[25(OH)D₃], E₂, and FSH. Total cholesterol, high-density lipoprotein cholesterol, low-density lipoprotein cholesterol, and triglyceride levels will be measured.

A 2-h fasting morning urine will be collected at the same time for measurements of pyridinium cross-links (pyridinoline [PYR] and deoxypyridinoline [DPYR]) and creatinine. These parameters will be evaluated at baseline and 6 months and 12 months after treatment.

Ca²⁺ (normal range, 2.1-2.6 mM) and creatinine (0.13-0.22 mmol kg⁻¹ of body weight/24 h in urine) will be determined by automated routine procedures. BGP (normal range, 1.6-17.4 ng/ml), B-ALP (normal range, 8.5-17.9 μg/liter), PTH (normal range, 1.2-7.2 pmol/liter), 25(OH)D₃ (normal range, 25-125 nM), and FSH (normal range, 21-153 IU/liter in postmenopausal phase) will be measured by a standard immunoradiometric methods. E₂ (normal range, 37-110 pmol/liter in postmenopausal phase) will be evaluated using a standary solid-phase immunoassay. PYR (normal range, 25-91 pmol/pmol of urinary creatinine) and D-PYR (normal range, 3-21 pmol/μmol of urinary creatinine) will be measured by an HPLC technique.

Clinic Visits:

The women will be questioned about any symptoms at clinic visits every 3 months. The subjects will complete the quality of life questionnaire documenting the frequency and severity of menopausal symptoms. Standard clinical evaluations and laboratory analyses, including hematological, renal, and liver function tests, will be performed every 6 months. Transvaginal uterine ultrasound and PAP tests will be performed at baseline and after 6 months and 12 months, whereas mammography will be performed at baseline and after 1 year. All unfavorable and unintended clinical effects will be considered adverse effects and will be evaluated by the investigators with respect to severity, duration, seriousness, and relation to the study drugs and outcome.

Statistics:

The primary evaluation of the efficacy data according to the intention to treat will include all of the postmenopausal women in whom BMD was measured at baseline and after 1 year of treatment.

The effect of treatment on BMD will be assessed by ANOVA. The evaluation of the incidence of side effects and menopausal symptoms in the several groups of postmenopausal women will be carried out with the Fisher's exact probability test. All statistical tests will be two sided. All data will be reported as means and SD. A value of p<0.05 will be considered statistically significant. Stepwise linear regression will be used to select the independent predictors of increased BMD for the final best multivariate models. Variables will be included in the final model if the value of p<0.05.

Anticipated Results:

It is expected that both DIM and genistein treatment groups will show beneficial effects on bone health. The treatment response to combined treatment with DIM and genestein is expected to be greater that treatment with DIM or genistein alone. Only the DIM group and DIM/genistein combined group are expected to show significant improvement in menopausal symptoms.

What is claimed is:

1. A method for preventing or treating oral mucosal disorders, or a symptom thereof, comprising administering to a subject in need thereof a therapeutically effective amount of Diindolylmethane (DIM) or a DIM-related indole.

2. The method of claim 1, wherein the administering is by systemic administration

3. The method of claim 1, wherein the administering is by topical oral administration.

4. The method of claim 1, wherein the symptom is calculus accumulation, apthous ulcer, oral malodor, gingivitis, periodontitis, alveolar bone loss, or loosening of teeth.

5. The method of claim 1, wherein the subject is a human.

6. The method of claim 1, where the DIM-related indole is selected from the group consisting of:
a compound of formula I: 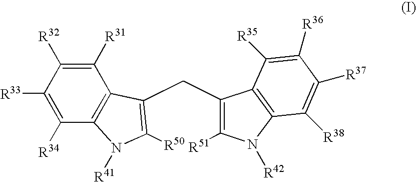
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 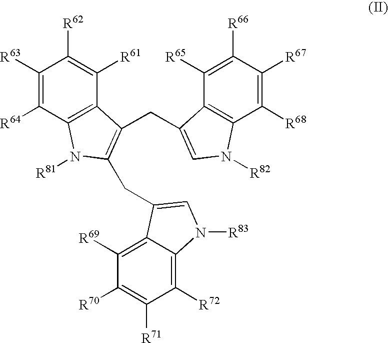
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 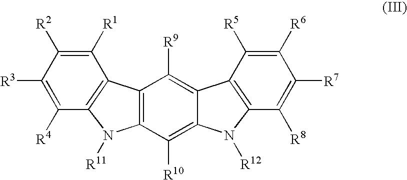
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl, with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are select from hydrogen, halo, alkyl and alkoxy, then R¹¹ and R¹² are other than hydrogen and alkyl;
a compound of formula (IV): 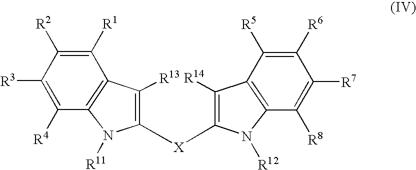
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 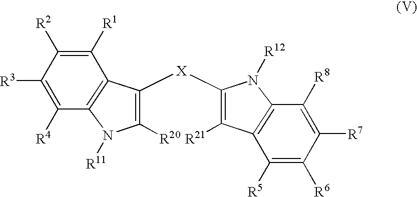
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

7. The method of claim 1, where the DIM or DIM-related indole is selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

8. The method of claim Error! Reference source not found., wherein the therapeutically effective amount of DIM or DIM-related indole is between 25-750 mg/day.

9. The method of claim Error! Reference source not found., wherein the therapeutically effective amount of DIM or DIM-related indole is between 50-200 mg/day.

10. The method of claim 3, wherein the therapeutically effective amount of DIM or DIM-related indole is between 20-60 mg/day.

11. The method of claim 3, wherein the therapeutically effective amount of DIM or DIM-related indole is between 20-30 mg/day.

12. The method of claim 1, wherein the DIM or DIM-related indole is formulated in a toothpaste, oral gel, mouth wash, tooth powder, dental floss, or chewing gum.

13. The method of claim 1, wherein the DIM or DIM-related indole is microencapsulated with tocopheryl succinate polyethylene glycol 1000 (TPGS) in a capsule or tablet.

14. The method of claim 1, wherein the DIM or DIM-related indole is microencapsulated and complexed with beta cyclodextrin, hydroxypropylmethylcellulose, chitosan or beta-1,3-glucan.

15. The method of claim 1, comprising administering an anti-inflammatory compound.

16. The method of claim 15, wherein the anti-inflammatory compound is resveratrol, an extract of Polygonium cuspidatum, silibinin, an extract of Silybum marianum, curcumin, an extract of Curcuma domestica, aloe extract, terpene, citrus extract, boswellic acid, Evodiamine, ursolic acid, Allyl Disulfide, Andrographolide, Dehydro-Andrographolide, Deoxy-Andrographolide, Brassinin, Caffeic acid, Capsanthin, Capsaincin, L-Carnitine, L-Carnitine HCl, Carnosic acid, Chelerythrine Chloride, Cromolyn sodium, Diallyl disulfide, Diallyl sulfide, Diallyl trisulfide, Dibenzoylmethane, Ebulin 1, Ellagic acid, (−)Epicatechin, (−)Epicatechin gallate, (−)Epigallocatechin, Epigallocatechin gallate, Ferulic acid, Genistein, 18β-Glycyrrhetinic Acid, Glycyrrhizic acid ammonium salt trihydrate, a Green tea polyphenol, Honokiol, 5-Hydroxy-L-tryptophan, Hypericin, Hypocrellin A, Ibuprofen, Idebenone, D-Limonene, Limonin, Limonin Glucoside, DL-α-Lipoic acid, Melatonin, Perillyl Alcohol, Phenylbutyrate, Phenylethyl 3-mehtylcaffeate, Phenylethyl 4-methylcaffeate, Phenyl isothiocyanate, Phytic Acid, 9-cis-Retinoic acid, 13-cis-Retinoic acid, trans-Retinoic acid, all-trans-Retinol, retinyl acetate, Retinyl palmitate, Rosmarinic acid, Rutaecarpine, L-Theanine, Trichostatin A, Vitamin K3, flurbiprofen, ketoprofen, aspirin, salycylamide, kertorolac, naproxen, indomethacin, piroxicam, meclofenamic acid, N-Acetyl-L-Cysteine, Zinc citrate, or Zinc gluconate.

17. The method of claim 15, wherein the anti-inflammatory compound is Caffeic acid, caffeic acid phenethyl ester (CAPE), an extract of the leaves of the Simon sweet potato, parthenolide, an extract of Tanacetum parthenium, an extract from Elder Flower or Elderberry, a citrus flavonoid, deguelin, sulforaphane, or xylitol.

18. The method of claim 17, wherein the citrus flavinoid is hesperidin, naringin, nobiletin, luteolin or apigenin.

19. The method of claim 15, wherein the DIM, or DIM-related indole, and anti-inflammatory compound are administered simultaneously.

20. The method of claim 15, wherein the DIM, or DIM-related indole, and anti-inflammatory compound are administered within a short time of one another.

21. The method of claim 15, wherein the DIM, or DIM-related indole, and anti-inflammatory compound are formulated in a toothpaste, oral gel, mouth wash, tooth powder, dental floss, or chewing gum.

22. The method of claim 1, comprising administering an antibacterial compound.

23. The method of claim 22, wherein the antibacterial compound is tea tree oil, neem oil, manuka oil, eucalyptus oil, lavandula oil, rosmarinus oil, rosmarinic acid, an aloe extract, a green tea extract, a perilla seed extract, a grapefruit seed extract, a Magnolia Grandiflora Seed Extract, Stevia extract, an extract of Prunella vulgaris, an Isoquinoline alkaloid from Macleya cordata, chitosan, triclosan, sanguinarine, sanguinaria, a quaternary ammonium compound, cetylpyridinium chloride, tetradecylpyridinium chloride and N-tetradecyl-4-ethylpyridinium chloride, benzalkonium chloride, a bisquanide, chlorhexidine, chlorhexidine digluconate, hexetidine, octenidine, alexidine, a halogenated bisphenolic compound, 2,2′-methylenebis-(4-chloro-6-bromophenol), 5-chloro-2-(2,4-dichloropheno-xy)-phenol, salicylanilide, domiphen bromide, delmopinol, octapinol, other piperadino derivatives, niacin, a zinc stannous ion agent, or an analog or salt of the foregoing.

24. The method of claim 22, wherein the DIM, or DIM-related indole, and antibacterial compound are administered simultaneously.

25. The method of claim 22, wherein the DIM, or DIM-related indole, and antibacterial compound are administered within a short time of one another.

26. The method of claim 22, wherein the DIM, or DIM-related indole, and anti-inflammatory compound are formulated in a toothpaste, oral gel, mouth wash, tooth powder, or chewing gum.

27. A composition comprising DIM, or a DIM-related indole, in an amount effective to reduce oral mucosal inflammation, formulated in the form of a toothpaste, oral gel, mouth wash, dental floss, or chewing gum.

28. The composition of claim 27, where the DIM-related indole is selected from the group consisting of:
a compound of formula I: 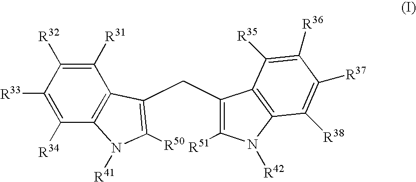
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 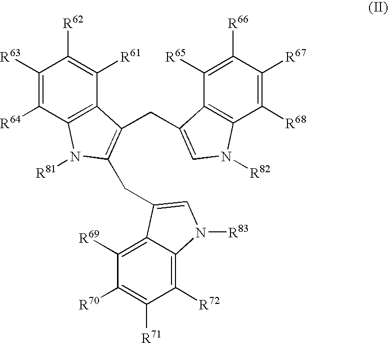
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 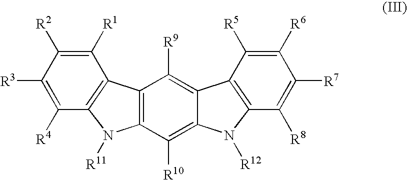
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl, with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 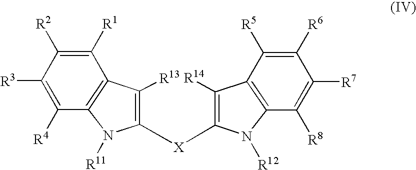
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino; R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 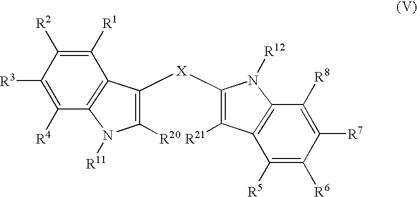
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

29. The composition of claim 27, where the DIM or DIM-related indole is selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

30. The composition of claim 27, wherein the DIM, or DIM-related indole, is microencapsulated with TPGS.

31. The composition of claim 27, wherein the DIM, or DIM-related indole, is microencapsulated and complexed with beta cylodextrin.

32. The composition of claim 27, wherein the composition is in the form of a toothpaste.

33. The composition of claim 27, wherein the composition is in the form of an oral gel.

34. The composition of claim 27, wherein the composition is in the form of a mouthwash.

35. The composition of claim 27, wherein the composition is in the form of a tooth powder.

36. The composition of claim 27, wherein the composition is in the form of a chewing gum.

37. The composition of claim 27, wherein the composition is in the form of a dental floss.

38. The composition of claim 27, comprising an anti-inflammatory compound.

39. The composition of claim 38, wherein the anti-inflammatory compound is resveratrol, an extract of Polygonium cuspidatum, silibinin, an extract of Silybum marianum, curcumin, an extract of Curcuma domestica, aloe extract, terpene, citrus extract, boswellic acid, Evodiamine, ursolic acid, Allyl Disulfide, Andrographolide, Dehydro-Andrographolide, Deoxy-Andrographolide, Brassinin, Caffeic acid, Capsanthin, Capsaincin, L-Carnitine, L-Carnitine HCl, Carnosic acid, Chelerythrine Chloride, Cromolyn sodium, Diallyl disulfide, Diallyl sulfide, Diallyl trisulfide, Dibenzoylmethane, Ebulin 1, Ellagic acid, (−)Epicatechin, (−)Epicatechin gallate, (−)Epigallocatechin, Epigallocatechin gallate, Ferulic acid, Genistein, 18β-Glycyrrhetinic Acid, Glycyrrhizic acid ammonium salt trihydrate, a Green tea polyphenol, Honokiol, 5-Hydroxy-L-tryptophan, Hypericin, Hypocrellin A, Ibuprofen, Idebenone, D-Limonene, Limonin, Limonin Glucoside, DL-α-Lipoic acid, Melatonin, Perillyl Alcohol, Phenylbutyrate, Phenylethyl 3-mehtylcaffeate, Phenylethyl 4-methylcaffeate, Phenyl isothiocyanate, Phytic Acid, 9-cis-Retinoic acid, 13-cis-Retinoic acid, trans-Retinoic acid, all-trans-Retinol, retinyl acetate, Retinyl palmitate, Rosmarinic acid, Rutaecarpine, L-Theanine, Trichostatin A, Vitamin K3, flurbiprofen, ketoprofen, aspirin, salycylamide, kertorolac, naproxen, indomethacin, piroxicam, meclofenamic acid, N-Acetyl-L-Cysteine, Zinc citrate, or Zinc gluconate.

40. The method of claim 38, wherein the anti-inflammatory compound is Caffeic acid, caffeic acid phenethyl ester (CAPE), an extract of the leaves of the Simon sweet potato, parthenolide, an extract of Tanacetum parthenium, an extract from Elder Flower or Elderberry, a citrus flavonoid, deguelin, sulforaphane, or xylitol.

41. The method of claim 40, wherein the citrus flavinoid is hesperidin, naringin, nobiletin, luteolin or apigenin.

42. The composition of claim 27, comprising administering an antibacterial compound.

43. The composition of claim 42, wherein the antibacterial compound is tea tree oil, neem oil, manuka oil, eucalyptus oil, lavandula oil, rosmarinus oil, rosmarinic acid, aloe extract, a green tea extract, a perilla seed extract, a grapefruit seed extract, a Magnolia Grandiflora Seed Extract, Stevia extract, an extract of Prunella vulgaris, an Isoquinoline alkaloid from Macleya cordata, chitosan, a chitosan derivative, triclosan, sanguinarine, sanguinaria, a sanguinarian extract, a quaternary ammonium compound, cetylpyridinium chloride, tetradecylpyridinium chloride and N-tetradecyl-4-ethylpyridinium chloride, benzalkonium chloride, a bisquanide, chlorhexidine, chlorhexidine digluconate, hexetidine, octenidine, alexidine, a halogenated bisphenolic compound, 2,2′-methylenebis-(4-chloro-6-bromophenol), 5-chloro-2-(2,4-dichloropheno-xy)-phenol, salicylanilide, domiphen bromide, honokiol, rosmarinic acid, delmopinol, octapinol, a piperadino derivative, niacin, a zinc stannous ion agent, or an analog or salt of the foregoing.

44. A method for preventing or treating bone loss or a symptom thereof, comprising administering to a subject in need thereof a therapeutically effective amount of (1) DIM or a DIM-related indole, and (2) genistein.

45. The method of claim 44, wherein the bone loss is characterized by diminished bone density on x-ray bone densitometry, loss of vertical height, alveolar bone loss on physical exam, or abnormal serum or urinary bone health markers.

46. The method of claim 44, wherein the subject is a mammal.

47. The method of claim 46, wherein the mammal is human.

48. The method of claim 44, where the DIM-related indole is selected from the group consisting of:
a compound of formula I: 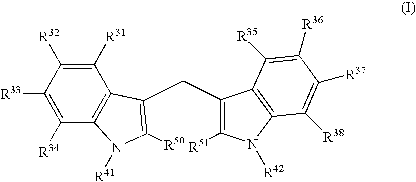
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 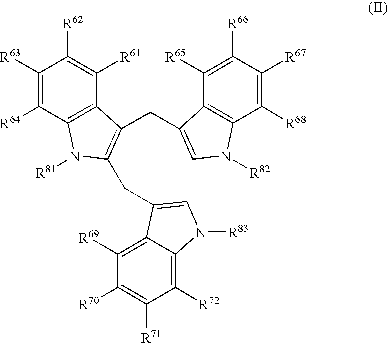
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸² and R⁸³ are hydrogen;
a compound of formula (III): 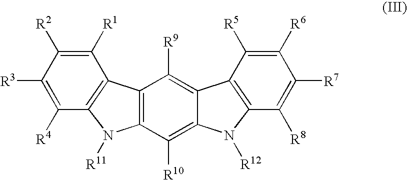
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl, with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 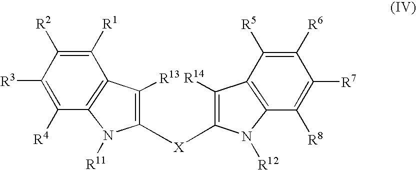
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 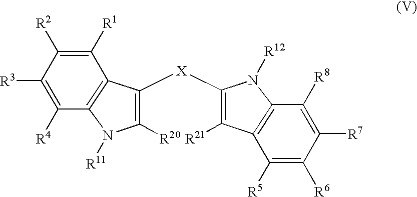
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

49. The method of claim 44, where the DIM or DIM-related indole is selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

50. The method of claim 44, wherein the administering of the DIM or DIM-related indole is by systemic or topical administration.

51. The method of claim 44, wherein the administering of genistein is by systemic or topical administration.

52. The method of claim 44, wherein the therapeutically effective amount of DIM or DIM-related indole is between 25-750 mg/day.

53. The method of claim 44, wherein the therapeutically effective amount of DIM or DIM-related indole is between 50-200 mg/day.

54. The method of claim 44, wherein the therapeutically effective amount of DIM or DIM-related indole is between 20-60 mg/day.

55. The method of claim 44, wherein the therapeutically effective amount of genistein is between 25-1,000 mg/day.

56. The method of claim 44, wherein the therapeutically effective amount of genistein is between 25-200 mg/day.

57. The method of claim 44, wherein the DIM,or DIM-related indole, and genistein are formulated in a toothpaste, oral gel, mouth wash, tooth powder, or chewing gum.

58. The method of claim 44, wherein the DIM or DIM-related indole is microencapsulated with phosphatidyl choline.

59. The method of claim 44, wherein the DIM or DIM-related indole is microencapsulated with tocopheryl succinate polyethylene glycol 1000 (TPGS) in a capsule or tablet.

60. The method of claim 44, wherein the DIM or DIM-related indole is microencapsulated and complexed with beta cyclodextrin, hydroxypropylmethylcellulose, chitosan or beta-1,3-glucan.

61. The method of claim 44, wherein the DIM-related indole and genistein are administered simultaneously.

62. The method of claim 44, wherein the DIM-related indole and genistein are administered within a short time of one another.

63. The method of claim 44, wherein the subject has an inflammatory condition.

64. The method of claim 63, wherein the inflammatory condition is rheumatoid arthritis or systemic lupus erythematosus.

65. A composition comprising (1) DIM, or a DIM-related indole; and (2) genistein, in an amount effective to reduce treat or prevent bone loss or a symptom thereof.

66. The composition of claim 65 formulated in the form of tablet, capsule, drink mix, fortified food, or chewing gum.

67. The composition of claim 66, wherein the fortified food is a Food Bar, Drink Mix, Vegetable Juice, Pasta Mix, Dry Cereal, Meal Replacement Powder, or Baked Good.

68. The composition of claim 65 formulated in the form of a topical gel, lotion, ointment, or cream.

69. The composition of claim 65, where the DIM-related indole is selected from the group consisting of:
a compound of formula I: 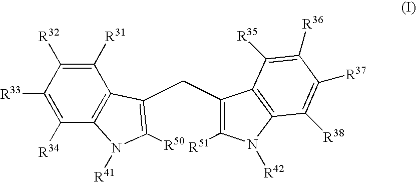
wherein R³² and R³⁶ are substituents independently selected from the group consisting of hydrogen, hydroxyl, and methoxy, and ethoxycarbonyl groups,
R³³ and R³⁷ are substituents independently selected from the group consisting of hydrogen, hydroxyl, and methoxy,
R³¹, R³⁴, R³⁵, R³⁸, R⁴¹, and R⁴² are hydrogen, and
R⁵⁰, R⁵¹ are either hydrogen or methyl;
a compound of formula II: 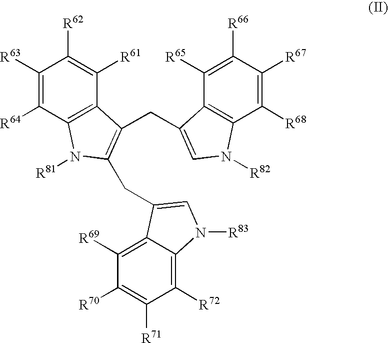
wherein R⁶², R⁶³, R⁶⁶, R⁶⁷, R⁷⁰, and R⁷¹ are substituents independently selected from the group consisting of hydrogen, hydroxyl, and methoxy, and
R⁶¹, R⁶⁴, R⁶⁵, R⁶⁸, R⁶⁹, R⁷², R⁸¹, R⁸², and R⁸³ are hydrogen;
a compound of formula (III): 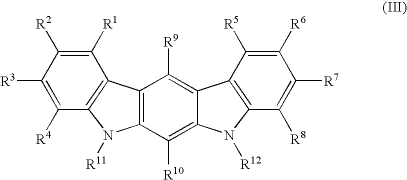
wherein R¹, R², R³, R⁴, R⁵, R⁶, R⁷, R⁸, R⁹, and R¹⁰ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, and
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl, with the provisos that at least one of R¹, R², R³, R⁴, R⁵, R⁶, R⁷, R⁸, R⁹, R¹⁰, R¹¹ and R¹² is other than hydrogen, and when R¹, R², R³, R⁴, R⁵, R⁶, R⁷, and R⁸ are selected from hydrogen, halo, alkyl and alkoxy, then R¹¹ and R¹² are other than hydrogen and alkyl;
a compound of formula (IV): 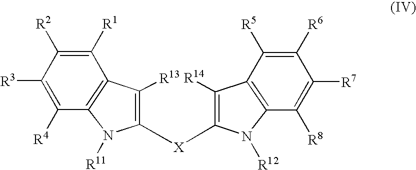
wherein R¹, R², R³, R⁴, R⁵, R⁶, R⁷, and R⁸ are substituents independently selected from the group consisting of hydrogen, C₁-C₂₄ alkyl, C₂-C₂₄ alkenyl, C₂-C₂₄ alkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄ alkyl)-substituted carbamoyl, di-(C₁-C₂₄ alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(C₁-C₂₄ alkyl)-substituted amino, mono- and di-(C₅-C₂₀ aryl)-substituted amino, C₂-C₂₄ alkylamido, C₅-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, phosphono, phosphonato, phosphinato, phospho, phosphino, and combinations thereof, and further wherein any two adjacent (ortho) substituents may be linked to form a cyclic structure selected from five-membered rings, six-membered rings, and fused five-membered and/or six-membered rings, wherein the cyclic structure is aromatic, alicyclic, heteroaromatic, or heteroalicyclic, and has zero to 4 non-hydrogen substituents and zero to 3 heteroatoms, with the proviso that one but not both of R² and R⁶ is amino, mono-substituted amino, or di-substituted amino;
R¹¹ and R¹² are independently selected from the group consisting of hydrogen, C₁-C₂₄ alkyl, C₂-C₂₄ alkoxycarbonyl, amino-substituted C₁-C₂₄ alkyl, (C₁-C₂₄ alkylamino)-substituted C₁-C₂₄ alkyl, and di-(C₁-C₂₄ alkyl)amino-substituted C₁-C₂₄ alkyl,
R¹³ and R¹⁴ are defined as for R¹, R², R³, R⁴, R⁵, R⁶, R⁷, and R⁸, with the proviso that at least one of R¹³ and R¹⁴ is other than hydrogen, and
X is O, S, arylene, heteroarylene, CR¹⁵R¹⁶ or NR¹⁷ wherein R¹⁵ and R¹⁶ are hydrogen, C₁-C₆ alkyl, or together form ═CR¹⁸R¹⁹ where R¹⁸ and R¹⁹ are hydrogen or C₁-C₆ alkyl, and R¹⁷ is as defined for R¹¹ and R¹²; and
a compound of formula (V): 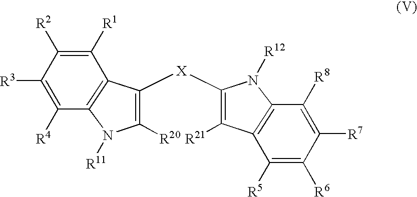
wherein R¹, R², R³, R⁴, R⁵, R⁶, R⁷, R⁸, R¹¹, R¹², and X are defined as for compounds of formula (III), and
R²⁰ and R²¹ are defined as for R¹, R², R³, R⁴, R⁵, R⁶, R⁷, and R⁸.

70. The composition of claim 65, where the DIM or DIM-related indole is selected from the group consisting of diindolylmethane, hydoxylated DIMs, methoxylated DIMs, 2-(Indol-3-ylmethyl)-3,3′-diindolylmethane (LTR), hydroxylated LTRs, methoxylated LTRs, 5,5′-dimethylDIM (5-Me-DIM), 2,2′-dimethylDIM (2-Me-DIM), 5,5′-dichloroDIM (5-Cl-DIM), imidazolelyl-3,3′-diindolylmethane, nitro-substituted imidazolelyl-3,3′-diindolylmethanes, 2,10-dicarbethoxy-6-methoxy-5,7-dihydro-indolo-[2,3-b]carbazole, 6-ethoxycarbonyloxy-5,7-dihydro-indolo-[2,3-b]carbazole and 2,10-dicarbethoxy-6-ethoxycarbonyloxy-5,7-dihydro-indolo-[2,3-b]carbazole, and 2,6-dicarbethoxy-3,3′-dimethyl-13,14-diindolylmethane.

71. The composition of claim 65, wherein the DIM, or DIM-related indole, and genistein are in particulate form.

72. The composition of claim 65, wherein the DIM, or DIM-related indole, and genistein are microencapsulated with phosphatidyl choline.

73. The composition of claim 65, wherein the DIM, or DIM-related indole, and genistein are microencapsulated with TPGS.

74. The composition of claim 65, wherein the DIM, or DIM-related indole, and genistein are microencapsulated and complexed with beta cylodextrin.
